(19) United States
(12) Patent Application Publication
(10) Pub. No.: US 2007/0081989 A1
(43) Pub. Date: Apr. 12, 2007

(54) Treatment of B cell diseases using anti-germline antibody binding agents

(76) Inventor: Martin E. Sanders, Hillsborough, CA (US)

Correspondence Address: CYNTHIA RUTH MOORE, 794 LOS ROBLES AVENUE, PALO ALTO, CA, 94306, US

(21) Appl. No.: 11/524,531

(22) Filed: Sep. 19, 2006

Related U.S. Application Data
(60) Provisional application No. 60/718,796, filed on Sep. 19, 2005.

(51) Int. Cl. A61K 39/395; G01N 33/567; A61K 39/00

(52) U.S. Cl. 424/131.1; 435/7.2; 424/140.1

(57) ABSTRACT

Methods for reducing the number of pathologic antibody producing B cells in a patient suffering from an autoimmune disease by administration of an anti-germline antibody are described. Methods for removing pathologic antibodies and B cells and plasma cells producing pathologic antibodies from the body of a patient suffering from autoimmune disease are provided, comprising contacting the blood or plasma of the patient with an immunoadsorbent having specific binding for an epitope present on germline antibodies, particularly VH4-34 antibodies, wherein said contacting results in the reduction in the amount of germline antibodies present in the blood or bone marrow or lymphoid tissue of the patient or the amount of germline antibody producing B cells present in the blood, lymphoid tissues or bone marrow of the patient. Methods for treating a patient suffering from a B cell cancer expressing cell surface germline antibodies by similar methods are also provided. Methods for ex vivo purging bone marrow of pathologic antibody producing B-cells and cancerous B-cells expressing germline antibodies are provided. Methods for monitoring the efficacy of a therapeutic treatment in a patient suffering from an autoimmune disease or B cell cancer are also provided. Kits and uses in preparation of a medicament are also described.

### CROSS REFERENCE TO RELATED APPLICATIONS

This application claims benefit of U.S. Provisional Patent Application No. 60/718,796, filed Sep. 19, 2005, which is incorporated herein by reference.

### FIELD OF THE INVENTION

This invention relates generally to methods for treating B cell diseases such as cancers or autoimmune diseases and the like.

### BACKGROUND OF THE INVENTION

Germline antibodies (i.e., antibodies having a high amino acid sequence homology to antibodies encoded by genomic DNA sequences in the absence of somatic hypermutation) have been associated with autoimmune disease and leukemias and lymphomas. For example, some authors have reported that the VH1-69 gene was shown to be associated with unmutated chronic lymphocytic leukemia (CLL) cases (Brezinschek H. P., et al. (1998) Br. J. Haematol. 102, 516-521). Similarly, Guarini, A. et al. ((2003) Blood 102, 1035-41) reported that this gene was not observed in a patient population exhibiting stable leukemias having a favorable clinical prognosis. In addition, Bando Y, et al. ((2004) Immunol. Lett. 94, 99-106) found highly homologous VH1-69 derived sequences from patients suffering from allergic diseases, suggesting that a rather limited VH gene might be rearranged for specific IgE in early life.

Other germline antibodies associated with autoimmune disease and cancers include the VH4-34 gene encoded antibodies. As reported by Milner, et al., B cells expressing these antibodies represent a large fraction of the primary pre-antigenic repertoire (approx 5-10% of mature naïve B cells) (Milner, E. C. B., et al. (2005) Semin. Immun. 26, 433-452). Pugh-Bernard, et al. have suggested that the highly regulated expression of the VH4-34 antibodies may provide homeostasis of the immune system in order to prevent autoimmune disease. (Pugh-Bernard, A. E., et al. (2001) J. Clin. Invest. 108, 1061-1070). VH4-34 antibodies are autoreactive in the absence of somatic mutation and independent of the antibody light chain, and are elevated in patients with active systemic lupus erythematosis (SLE). In patients exhibiting symptoms of SLE, the presence of VH4-34 IgG and the absence of VH4-34 IgM antibodies were most strongly correlated with severity of SLE, nephritis and central nervous system lupus. (Bhat, N. M., et al. (2002) J. Rheumatol. 29, 2114-21). As described in WO 99/01477, patients suffering from autoimmune diseases, particularly SLE patients having severe SLE, were found to have levels of VH4-34 antibody that were significantly above those found in healthy individuals. These antibodies are also implicated in B cell cancers such as nodal marginal zone B cell lymphoma Marasca, R. et al., (2001) Am. J. Pathol. 159, 253-262.

Some researchers have suggested that the VH4-34 antibodies potentially provide beneficial or regulatory effects. One mechanism proposed for this beneficial effect focused on the crossreactivity of the VH4-34 antibodies with other nonprotein antigens such as bacterial LPS, DNA or tumor gangliosides. These authors proposed that under conditions of strong stimulation, the cells having surface VH4-34 antibodies could differentiate into marginal zone (MZ) cells and play a protective role either by clearing self-antigens by reacting with bacterial antigens or by maintaining peripheral T cell tolerance through the presentation of self-antigens. Pugh-Bernard, A. E., et al., (2001) J. Clin. Invest. 108, 1061-1070.

Other authors suggest there may be a pathogenic effect of VH4-34 antibodies in autoimmune diseases. Pathogenic effects were proposed as mediated by anti-dsDNA and anti-Smith responses, killing naive lymphocytes or promoting their differentiation into class-switched B cells, contributing to the sustained production of IFN-alpha, modulating the activation threshold of B cells, or by binding to gangliosides and contributing to neuropsychiatric SLE. Analik, J. and Sanz, I. (2004) Curr. Op. Rheumatol. 16, 505-512; Milner, E. C. B., et al. (2005) Semin. Immun. 26, 433-452.

Thus, the roles of these antibodies in protection against or mediating disease are unelucidated. Further, there is no teaching or suggestion that removal of these antibodies or the cells producing them could provide a beneficial result to patients suffering from autoimmune diseases or B cell cancers.

### SUMMARY OF THE INVENTION

Accordingly, the aforementioned need in the art is addressed by providing novel methods and therapeutic compositions for treating patients suffering from B cell diseases such as B cell cancers and autoimmune diseases and disorders.

In an additional embodiment, the aforementioned need in the art is addressed by providing novel methods and therapeutic compositions for treating patients suffering from B cell cancers expressing germline antibodies, such as VH4-34 antibodies.

Accordingly, in one embodiment, a method is provided for reducing the amount of B cells or plasma cells producing pathologic antibodies in the body of a patient suffering from an autoimmune disease, comprising treating the patient with a therapeutically effective dose of an antibody having specific binding for an epitope present on germline antibodies. Preferably, the germline antibodies are selected from VH4-34, VH1-69, V71-2, V71-4, VH4-18, VH72-1, or V2-1 antibodies. In a particular embodiment, the antibody having specific binding for an epitope present on germline antibodies is 9G4, humanized 9G4, chimerized 9G4, or fragments or conjugates thereof. In additional embodiments, the antibody having specific binding for an epitope present on germline antibodies is 9G4, G6, 17.109, or LC1, humanized 9G4, G6, 17.109, or LC1, chimerized 9G4, G6, 17.109, or LC1, or fragments or conjugates thereof. The method can further comprise treating the patient with an additional pharmaceutically active agent, therapeutically effective treatment or other adjunct therapy. Additional pharmaceutically active agents include chemotherapeutic agents, complement activation inhibitors, antimetabolites, steroids, toleragens, anti-B cell agents, anti-T cell agents, anticoagulants or intravenous immunoglobulin.

In another embodiment, a method is provided for reducing the amount of VH4-34 antibody producing B cells or plasma cells in a patient suffering from an autoimmune disease, comprising administering a therapeutically effective amount of an antibody having specific binding for an epitope present on VH4-34 antibodies. Preferably, the antibody having specific binding for an epitope present on VH4-34 antibodies is 9G4, humanized or chimerized 9G4, or fragments or conjugates thereof.

In an additional embodiment, a method is provided for treating a patient suffering from a B cell cancer expressing cell surface germline antibodies, comprising treating the patient with a therapeutically effective dose of an antibody having specific binding for an epitope present on germline antibodies. Preferably, the germline antibodies are selected from VH4-34, VH1-69, V71-2, V71-4, VH4-18, VH72-1, or V2-1 antibodies, and more preferably, the germline antibodies are VH4-34 antibodies. The antibody having specific binding for an epitope present on germline antibodies is preferably 9G4, humanized or chimerized 9G4, or fragments or conjugates thereof. In additional embodiments, the antibody having specific binding for an epitope present on germline antibodies is 9G4, G6, 17.109, or LC1, humanized 9G4, G6, 17.109, or LC1, chimerized 9G4, G6, 17.109, or LC1, or fragments or conjugates thereof. The method can further comprise treating the patient with an additional pharmaceutically active agent selected from a chemotherapeutic agent, anti-B cell agent, cell growth regulator and/or inhibitor, immune modulator or combinations thereof. Preferably, the chemotherapeutic agent is asparaginase, epipodophyllotoxin, camptothecin, antibiotic, platinum coordination complex, alkylating agent, folic acid analog, pyrimidine analog, purine analog, topoisomerase inhibitor, or an agent that disrupts the cytoskeleton, or mixtures thereof. preferred anti-B cell agent include antibodies or inhibitors of CD11a, CD19, CD20, CD21, CD22, CD25, CD34, CD37, CD38, CD40, CD45, CD52, CD80, CD 86, IL-4R, IL-6R, IL-8R, IL-13, IL-13R, α-4/β-1 integrin (VLA4), BLYS receptor, cell surface idiotypic Ig, CDIM, tumor necrosis factor (TNF), or combinations thereof. In a particular embodiment, the anti-B cell agent is an anti-CDIM antibody. The anti-CDIM antibody can be mAb 216, RT-2B, FS 12, A6(H4C5), Cal-4G, S20A2, FS 3, Gee, HT, Z2D2, or Y2K.

In alternative embodiments, methods are provided for purging the bone marrow of a patient suffering from autoimmune disease or B cell cancer prior to reimplantation of the bone marrow in the patient after myeloablative therapy, comprising treating the bone marrow of a patient ex vivo with a therapeutically effective amount of an antibody having specific binding for an epitope present on germline antibodies. The method can further comprise treating the bone marrow with an additional pharmaceutically active agent. Preferably, the antibody having specific binding for an epitope present on germline antibodies is 9G4, humanized or chimerized 9G4, or fragments or conjugates thereof. In additional embodiments, the antibody having specific binding for an epitope present on germline antibodies is 9G4, G6, 17.109, or LC1, humanized 9G4, G6, 17.109, or LC1, chimerized 9G4, G6, 17.109, or LC1, or fragments or conjugates thereof.

In another embodiment, a method is provided for purging the bone marrow of a patient suffering from autoimmune disease or B cell cancer prior to reimplantation of the bone marrow in the patient after myeloablative therapy, comprising treating the bone marrow of a patient ex vivo with a therapeutically effective amount of an antibody having specific binding for an epitope present on VH4-34 antibodies. The method can further comprise treating the bone marrow with an additional pharmaceutically active agent. The antibody having specific binding for an epitope present on VH4-34 antibodies is preferably 9G4, humanized or chimerized 9G4, or fragments or conjugates thereof.

In yet another embodiment, methods are provided for treating a patient suffering from autoimmune disease or a B cell cancer, comprising treating the patient with a therapeutically effective amount of an antibody having specific binding for an epitope present on germline antibodies, and further comprising treating the patient with a therapeutically effective amount of an anti-B cell agent. Preferably, the anti-B cell agent is an anti-CDIM antibody, selected from mAb 216, RT-2B, FS 12, A6(H4C5), Cal-4G, S20A2, FS 3, Gee, HT, Z2D2, or Y2K. In an additional aspect, the method includes providing sufficient time to allow the antibody having specific binding for an epitope present on germline antibodies to clear from the plasma of the patient prior to administering the anti-CDIM antibody. In another aspect, the method includes providing sufficient time to allow the anti-CDIM antibody to clear from the plasma of the patient prior to administering the antibody having specific binding for an epitope present on germline antibodies. A sufficient time generally is provided in 5 serum half-lives.

In yet other embodiments, methods are provided for removing pathologic antibodies from the body of a patient suffering from autoimmune disease, comprising contacting the blood or plasma of the patient with an immunoadsorbent having specific binding for an epitope present on germline antibodies. Preferably, the immunoadsorbent having specific binding for an epitope present on VH4-34 antibodies comprises 9G4, G6, 17.109, or LC1, humanized 9G4, G6, 17.109, or LC1, chimerized 9G4, G6, 17.109, or LC1, or fragrnents or conjugates thereof. The contacting preferably results in a reduction in the amount of germline antibodies present in the patient and or a reduction in the number of cells expressing germline antibodies in the patient. Preferably, the germline antibodies are selected from VH4-34, VH1-69, V71-2, V71-4, VH4-18, VH72-1, or V2-1 antibodies.

In another embodiment methods for removing pathologic antibodies from the body of a patient suffering from autoimmune disease are provided, comprising contacting the blood or plasma of the patient with an immunoadsorbent having specific binding for an epitope present on VH4-34 antibodies, wherein said contacting results in the reduction in the amount of VH4-34 antibodies present in the blood or plasma of the patient. Preferably, the immunoadsorbent has specific binding for an epitope present on VH4-34 antibodies comprises 9G4, humanized or chimerized 9G4, or fragments or conjugates thereof.

In another embodiment, methods are provided for reducing the number of VH4-34 antibody producing B cells or plasma cells in a patient suffering from an autoimmune disease, comprising contacting the blood or plasma of the patient with an immunoadsorbent having specific binding for an epitope present on VH4-34 antibodies, wherein said contacting results in the reduction in the amount of VH4-34 antibody producing B cells present in the blood, lymphoid tissues or bone marrow of the patient. Preferably, the immunoadsorbent has specific binding for an epitope present on VH4-34 antibodies comprises 9G4, humanized or chimerized 9G4, or fragments or conjugates thereof.

In an additional embodiment, a method is provided for treating a patient suffering from cold agglutinin disease, comprising treating the patient with a therapeutically effective dose of an antibody having specific binding for an epitope present on germline antibodies. Preferably, the germline antibodies are selected from VH4-34, VH1-69, V71-2, V71-4, VH4-18, VH72-1, or V2-1 antibodies. In another preferred embodiment, the germline antibodies comprise Vkappa I, Vkappa III or Vkappa IV light chains. In a particular embodiment, the germline antibodies are VH4-34 antibodies. In a preferred embodiment, the antibody having specific binding for an epitope present on germline antibodies is 9G4, humanized or chimerized 9G4, or fragments or conjugates thereof.

In additional embodiments, methods are provided for treating a patient suffering from a B cell cancer expressing cell surface germline antibody, comprising contacting the blood of the patient with an immunoadsorbent having specific binding for an epitope present on germline antibodies, wherein said contacting results in the reduction in the amount of germline antibody expressing B cell cancer cells present in the blood, lymphoid tissues or bone marrow of the patient. The methods can further comprise administering a therapeutically effective amount of an antibody having specific binding for an epitope present on germline antibodies to the patient. Preferably, the antibody having specific binding for an epitope present on germline antibodies is 9G4, humanized or chimerized 9G4, or fragments or conjugates thereof. In additional aspects, the antibody having specific binding for an epitope present on germline antibodies is 9G4, G6, 17.109, or LC1, humanized 9G4, G6, 17.109, or LC1, chimerized 9G4, G6, 17.109, or LC1, or fragments or conjugates thereof. The method can further comprise administering a therapeutically effective amount of an anti-CDIM antibody to the patient, such as mAb 216, RT-2B, FS 12, A6(H4C5), Cal-4G, S20A2, FS 3, Gee, HT, Z2D2, or Y2K.

In other embodiments, methods are provided for treating a patient suffering from a B cell cancer expressing cell surface VH4-34 antibody, comprising contacting the blood of the patient with an immunoadsorbent having specific binding for an epitope present on VH4-34 antibodies, wherein said contacting results in the reduction in the amount of VH4-34 antibody expressing B cell cancer cells present in the blood, lymphoid tissues or bone marrow of the patient. The methods can further comprise administering a therapeutically effective amount of an antibody having specific binding for an epitope present on VH4-34 antibodies to the patient. Preferably, the antibody having specific binding for an epitope present on VH4-34 antibodies is 9G4, humanized or chimerized 9G4, or fragments or conjugates thereof. The methods can further comprise administering a therapeutically effective amount of an anti-CDIM antibody to the patient, including mAb 216, RT-2B, FS 12, A6(H4C5), Cal-4G, S20A2, FS 3, Gee, HT, Z2D2, or Y2K.

In other embodiments, methods for monitoring the efficacy of a therapeutic treatment in a patient suffering from an autoimmune disease or a B cell cancer, comprising obtaining a sample of blood or bone marrow or lymphoid tissue from the patient, contacting said sample with an amount of anti-germline antibody sufficient to bind to germline antibodies present in the sample of blood or bone marrow or lymphoid tissue, determining the amount of anti-germline antibody bound in the sample of blood or bone marrow or lymphoid tissue, and correlating the amount of anti-germline antibody bound with the efficacy of treatment to reduce the number of germline antibody producing or cell surface expressing B cells or the amount of germline antibody in the sample of blood or bone marrow or lymphoid tissue obtained from the patient at a time prior to initiation of the therapeutic treatment. Preferably, the anti-germline antibody is 9G4, humanized or chimerized 9G4, or fragments or conjugates thereof. In other preferred embodiments, the anti-germline antibody is 9G4, G6, 17.109, or LC1, humanized 9G4, G6, 17.109, or LC1, chimerized 9G4, G6, 17.109, or LC1, or fragments or conjugates thereof. The anti-germline antibody can be associated with a substrate for performing an assay selected from ELISA or radioimmunoassay, or it can be utilized in flow cytometry.

The therapeutic treatments can include plasmapheresis, leukopheresis, or treatment with an anti-germline antibody or additional pharmaceutically active agent comprising an anti-B cell agent, anti-T cell agent, chemotherapeutic agent, toleragen, complement activation inhibitor, antimetabolite, steroid, anticoagulant or intravenous immunoglobulin, or combinations thereof.

In additional embodiments, methods are provided for monitoring the efficacy of a therapeutic treatment in a patient suffering from an autoimmune disease or a B cell cancer, comprising obtaining a sample of blood or bone marrow or lymphoid tissue from the patient, contacting said sample with an amount of anti-VH4-34 antibody sufficient to bind to VH4-34 antibodies present in the sample of blood or bone marrow or lymphoid tissue, determining the amount of anti-VH4-34 antibody bound in the sample of blood or bone marrow or lymphoid tissue, and correlating the amount of anti-VH4-34 antibody bound with the efficacy of treatment to reduce the number of VH4-34 antibody producing or cell surface expressing B cells or the amount of VH4-34 antibody in the sample of blood or bone marrow or lymphoid tissue obtained from the patient at a time period prior to initiation of the therapeutic treatment. Preferably, the anti-VH4-34 antibody is 9G4, humanized or chimerized 9G4, or fragments or conjugates thereof. The anti-VH4-34 antibody can be associated with a substrate for performing an assay selected from ELISA or radioimmunoassay, or utilized in flow cytometry. Preferably, the therapeutic treatment is plasmapheresis, leukopheresis, or treatment with an additional pharmaceutically active agent comprising an anti-B cell agent, anti-T cell agent, chemotherapeutic agent, toleragen, complement activation inhibitor, antimetabolite, steroid, anticoagulant or intravenous immunoglobulin.

In additional aspects, a kit is provided for use in monitoring the therapeutic response in a patient in need thereof to administration of a treatment for autoimmune disease or B cell cancer, comprising an amount of anti-germline antibody effective to bind to germline antibodies present in a sample of blood or bone marrow or lymphoid tissue from a patient suffering from said autoimmune disease or B cell cancer. Kits are also provided for use in monitoring the therapeutic response in a patient in need thereof to administration of a treatment for autoimmune disease or B cell cancer, comprising an amount of anti-VH4-34 antibody effective to bind to VH4-34 antibodies present in a sample of blood or bone marrow or lymphoid tissue from a patient suffering from said autoimmune disease or B cell cancer. In a particular embodiment, the kit comprises an amount of 9G4, humanized or chimerized 9G4, or fragments or conjugates thereof, effective to bind to VH4-34 antibodies present in a sample of blood or bone marrow or lymphoid tissue from a patient suffering from said autoimmune disease or B cell cancer.

In an additional aspect, the use of an anti-germline antibody in the manufacture of a medicament for the treatment of autoimmune disease or B cell cancer is provided. Preferably, the anti-germline antibody is an anti-VH4-34 antibody, more preferably, 9G4, humanized or chimerized 9G4, or fragments or conjugates thereof. In additional embodiments, the anti-germline antibody is 9G4, G6, 17.109, or LC1, humanized 9G4, G6,17.109, or LC1, chimerized 9G4, G6, 17.109, or LC1, or fragments or conjugates thereof.

In yet another aspect, an immunoadsorbent is provided for use in plasmapheresis and leukopheresis, comprising an anti-germline antibody or fragment thereof associated with a sorbent suitable for use in a plasmapheresis or leukopheresis apparatus. Preferably, the anti-germline antibody is selected from an antibody having specific binding for VH4-34, VH1-69, V71-2, V71-4, VH4-18, VH72-1, or V2-1 antibodies, and more preferably, an anti-VH4-34 antibody such as 9G4, humanized or chimerized 9G4, or fragments or conjugates thereof In additional embodiments, the anti-germline antibody is 9G4, G6, 17.109, or LC1, humanized 9G4, G6, 17.109, or LC1, chimerized 9G4, G6, 17.109, or LC1, or fragments or conjugates thereof.

Additional objects, advantages and novel features of the invention will be set forth in part in the description which follows, and in part will become apparent to those skilled in the art upon examination of the following, or may be learned by practice of the invention.

### DETAILED DESCRIPTION OF THE INVENTION

I. Definitions and Overview

Before the present invention is described in detail, it is to be understood that unless otherwise indicated this invention is not limited to specific antibodies, antibody fragments, or the like, as such may vary. It is also to be understood that the terminology used herein is for the purpose of describing particular embodiments only and is not intended to limit the scope of the present invention.

It must be noted that as used herein and in the claims, the singular forms “a,” “and” and “the” include plural referents unless the context clearly dictates otherwise. Thus, for example, reference to “an antibody” includes two or more antibodies; reference to “a pharmaceutical agent” includes two or more pharmaceutical agents, and so forth.

Where a range of values is provided, it is understood that each intervening value, to the tenth of the unit of the lower limit unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

The term “antibody” is used in the broadest sense and specifically covers intact natural antibodies (e.g., the antibody classes IgA, IgD, IgE, IgG, or IgM), monoclonal antibodies, polyclonal antibodies, multispecific antibodies (e.g. bispecific antibodies) formed from at least two intact antibodies, synthetic antibodies such as tetravalent antibodies, engineered antibody variants (such as mixtures of light chains and heavy chains from different antibody classes, variations in the number of antibody light and heavy chains, or the presence or absence of J chain), and antibody fragments, so long as they exhibit the desired biological activity. An exemplary species of engineered antibody variant is hexameric IgM). Human antibodies include antibodies made in nonhuman species. The term antibody also encompasses Ig molecules formed only from heavy chains, such as those obtained from Camelids, and described in U.S. Pat. Nos. 6,765,087 and 6,015,695 to Casterman, for example. The term antibody also encompasses fusion or chemical coupling (i.e., conjugation) of antibodies with cytotoxic or cell regulating agents.

For therapeutic applications and formulations, it is preferred that the antibody be cytolytic, i.e., the antibody exhibits complement mediated cytotoxicity. However, antibodies that utilize other mechanisms of cytotoxicity can also be utilized for therapeutic applications. For immunoadsorption applications (e,g, for plasmapheresis or leukopheresis), it is not necessary that the antibody be cytolytic or that the intact antibody be present, so long as a binding fragment can bind to germline antibody or germline antibody expressing or producing cells.

“Antibody fragments” comprise a portion of an intact antibody, preferably the antigen binding or variable region of the intact antibody. Examples of antibody fragments include Fab, Fab′, F(ab′)₂, and Fv fragments; diabodies; linear antibodies (Zapata, et al. (1995) Protein Eng. 8(10),1057-1062) single-chain antibody molecules; and multispecific antibodies formed from antibody fragments.

The term “blood” refers to all components of blood, including whole blood, serum, plasma, cell fractions, and the like.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. In addition to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al., Nature 256, 495 (1975), or may be made by recombinant DNA methods (see, e.g., U.S. Pat. No. 4,816,567). The “monoclonal antibodies” can also be isolated from phage antibody libraries using the techniques described in Clackson et al. (1991) Nature, 352, 624-628 and Marks et al., (1991) J. Mol. Biol. 222, 581-597, for example.

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al., (1984) Proc. Natl. Acad. Sci. USA, 81, 6851-6855).

“Humanized” forms of non-human (e.g., murine) antibodies are engineered antibodies wherein the antigen binding region of an immunoglobulin of non-human origin is incorporated into the antigen binding region of the parent human immunoglobulin. Humanized antibodies can include the natural antibody classes (IgA, IgD, IgE, IgG, or IgM), chimeric immunoglobulins, immunoglobulin chains or fragments thereof (such as Fv, Fab, Fab′, F(ab′)₂ or other antigen-binding subsequences of antibodies) which contain minimal sequence derived from non-human immunoglobulin. For the most part, humanized antibodies are human immunoglobulins in which residues from a complementarity determining region (CDR) are replaced by residues from a CDR of a non-human species such as mouse, rat or rabbit, etc. having the desired specificity, affinity, and capacity for a particular antigen of interest. In some instances, framework region (FR) residues of the human immunoglobulin are also replaced by corresponding non-human residues. Furthermore, humanized antibodies may comprise additional residues which are found neither in the parent antibody nor in the imported CDR or framework sequences. These modifications can be made to further refine and maximize antibody performance. In general, the humanized antibody will comprise substantially all of at least one, and typically two, variable domains, in which all or substantially all of the CDRs correspond to those of a non-human immunoglobulin and all or substantially all of the FRs are those of a human immunoglobulin sequence. The humanized antibody optimally also will comprise at least a portion of an immunoglobulin constant region (Fc), typically that of a human immunoglobulin. For further details, see Jones et al., (1986) Nature 321, 522-525; Reichmann et al., (1988) Nature 332, 323-329; and Presta (1992) Curr. Op. Struct. Biol. 2, 593-596. The humanized antibody includes a PRIMATIZED™ antibody wherein the antigen-binding region of the antibody is derived from an antibody produced by immunizing macaque monkeys with the antigen of interest.

“Single-chain Fv” or “scFv” antibody fragments comprise the VH and VL domains of antibody, wherein these domains are present in a single polypeptide chain. Preferably, the Fv polypeptide further comprises a polypeptide linker between the VH and VL domains which enables the scFv to form the desired structure for antigen binding. For a review of scFv see Pluckthun in The Pharmacology of Monoclonal Antibodies, vol. 113, Rosenburg and Moore eds., Springer-Verlag, New York, pp. 269-315 (1994).

The term “diabodies” refers to small antibody fragments with two antigen-binding sites, which fragments comprise a heavy-chain variable domain (VH) connected to a light-chain variable domain (VL) in the same polypeptide chain (VH-VL). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al. (1993) Proc. Natl. Acad. Sci. USA 90, 6444-6448.

An “isolated” antibody is one which has been identified and separated and/or recovered from a component of its natural environment. Contaminant components of its natural environment are materials which would interfere with diagnostic or therapeutic uses for the antibody, and may include enzymes, hormones, and other proteinaceous or nonproteinaceous solutes. In preferred embodiments, the antibody will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

The term “germline antibody” refers to antibodies having a high amino acid sequence homology to antibodies encoded by genomic DNA sequences in the absence of somatic hypermutation. Germline antibodies generally exhibit an amino acid sequence homology in the variable region compared to the amino acid sequence encoded by the closest germline gene of at least about 60%, preferably ranging from a sequence homology of about 60% to about 100%, or more preferably between about 75% and 99%. Such antibodies have undergone minimal or no somatic hypermutation, which is characteristic of nongermline antibodies.

The phrase “antibody having specific binding for an epitope on germline antibodies” or the term “anti-germline antibody” refers to an antibody (including intact antibodies, chimerized or engineered or fused antibodies, or fragments, conjugates, etc., thereof) that binds to an epitope on the variable region of an antibody encoded by germline DNA sequences.

The term “epitope” refers to a unique marker on the variable region of the antibody class encoded by the genomic DNA sequence of that antibody, and as such can include the germline sequence of the so called hypervariable regions or “complementarity determining regions” (“CDRs”) of the antibody. However, the epitope is not a marker of a unique immunoglobulin formed by somatic hypermutation, such as a nongermline CDR. Accordingly, the anti-germline antibody is not a “patient specific” antibody. See for contrast Timmerman, J. M. and Levy, R. (2000) Clin. Lymphoma 1, 129. Preferably, the epitope is present in the framework region of the antibody. Preferably, the epitope does not include the CDR of the antibody.

The “CD20” antigen is a 35 kDa, non-glycosylated phosphoprotein found on the surface of greater than 90% of B cells from peripheral blood or lymphoid organs. CD20 is expressed during early pre-B cell development and remains until plasma cell differentiation. CD20 is present on both normal B cells as well as malignant B cells. Other names for CD20 in the literature include “B-lymphocyte-restricted antigen” and “Bp35.” The CD20 antigen is described in Clark et al. (1985) PNAS (USA) 82, 1766, for example.

The term “conjugate” refers to coupling of active agents, which can be covalent or noncovalently associated.

A “disorder” is any condition that would benefit from treatment with the combination therapy described herein. This includes chronic and acute disorders or diseases including those pathological conditions which predispose the mammal to the disorder in question. Non-limiting examples of disorders to be treated herein include cancer, hematological malignancies, leukemias and lymphoid malignancies and autoimmune diseases such as inflammatory and immunologic disorders.

The term “pathologic antibodies” refers to antibodies exhibiting specific binding against self antigens, i.e., the antibodies are autoreactive. Such pathologic antibodies are implicated in or associated with autoimmune disorders.

The term “specific binding” refers the property of having a high binding affinity of at least 10⁶ M⁻¹, and usually between about 10⁶ M⁻¹ and about 10⁸ M⁻¹.

The term “therapeutically effective amount” is used to refer to an amount of an active agent having a growth arrest effect or causes the death of the cell. In certain embodiments, the therapeutically effective amount has the property of permeabilizing cells, inhibiting proliferative signaling, inhibiting cellular metabolism, modulating B cell function, promoting apoptotic activity, or inducing cell death. In particular aspects, the therapeutically effective amount refers to a target serum concentration that has been shown to be effective in, for example, slowing disease progression. Efficacy can be measured in conventional ways, depending on the condition to be treated. For example, in lymphoid cancers, efficacy can be measured by assessing the time to disease progression (TTP), or determining the response rates (RR). A therapeutically effective amount is also an amount sufficient to reduce the numbers of B cells producing genrline antibodies or to decrease the amount of germline antibody in the patient.

The terms “treat,” “treatment” and “therapy” and the like are meant to include therapeutic as well as prophylactic, or suppressive measures for a disease or disorder leading to any clinically desirable or beneficial effect, including but not limited to alleviation of one or more symptoms, regression, slowing or cessation of progression of the disease or disorder. Thus, for example, the term treatment includes the administration of an agent prior to or following the onset of a symptom of a disease or disorder thereby preventing or removing all signs of the disease or disorder. As another example, the term includes the administration of an agent after clinical manifestation of the disease to combat the symptoms of the disease. Further, administration of an agent after onset and after clinical symptoms have developed where administration affects clinical parameters of the disease or disorder, such as the degree of tissue injury or the amount or extent of metastasis, whether or not the treatment leads to amelioration of the disease, comprises “treatment” or “therapy” within the context of the invention.

The term “9G4” refers to the rat monoclonal antibody that has been shown to recognize VH4-34 antibody (Stevenson, et al. Blood 68: 430 (1986)). The VH4-34 epitope identified by mAb 9G4 is conformation restricted and dependent on a unique sequence near amino acids 23-25 in the framework 1 region (“FR1”) of the variable heavy chain. The VH4-34 gene has low incidence of mutation, allowing the reliable detection of VH4-34 antibodies using 9G4 by standard immunoassay methods.

The term “LC1” refers to an antibody that binds to antibodies derived from VH4 family gene segments V71-2, V71-4, VH4-18, VH72-1 and V2-1. The epitope of LC1 also maps to FR1. See Melero, J. et al. (1998) Scand. J. Immunol. 48, 152.

The VH4-34 antibodies (variable heavy region) are one of the 53 identified human functional antibody germline antibodies, and are encoded by germline genes (VH4.21). Cook, G. P., et al., (1994) Nat. Genet. 7, 162-168. The gene for VH4-34 antibodies is present in all haplotypes and no sequence variation has been reported in germline DNA isolated from unrelated individuals. Weng, N. P., et al., (1992) Eur. J. Immunol. 22,1075-1082; van der Maarel, S., et al., (1993) J. Immunol. 150, 2858-2868. Antibodies encoded by the VH4-34 gene have been shown to possess unique properties. All mabs directed against the “I” or “i” antigens of red blood cells (RBCs) are encoded by the VH4-34 gene, are generally of the IgM class, and are classically described as cold agglutinins (CAs) because they agglutinate RBCs at 4° C. Pascual, V., et al., (1991) J. Immunol. 146, 4385-4391; Pascual, V., (1992) J. Immunol. 149,2337-2344; Silberstein, L. E., et al., (1991) Blood 78, 2372-2386. The ligands recognized by CAs are linear or branched glycoconjugates present on proteins and/or lipids of the RBCs. Newborn and cord blood RBC possess the linear i antigen. The branched I chain is generated after birth. Pruzanski, W. et al., (1984) Clin. Immunol. Rev. 3,131-168; Roelcke, D. (1989) Transfusion Med. Rev. 2,140-166.

The “i” antigen recognized on human B cells is a linear lactosamine determinant that is sensitive to the enzyme endo-beta-galactosidase. Sequence analysis of independently derived VH4-34 anti-B cell/anti-i mAbs has shown that they are in germline configuration. Bhat N. M., et al., (1997) Clin. Exp. Immunol. 108,151-159. Cold agglutinins of anti-I/I specificity are restricted to VH4-34 heavy chain expression. Anti-Pr cold agglutinins recognize alpha 2,3- or alpha 2,6-linked N-acetylneuraminic acid. Cold agglutinins of anti-Pr specificity exhibit expression of the following light chains: Vkappa I, Vkappa III or Vkappa IV, with a preference for the use of the single germline gene-derived subgroup, Vkappa IV. Lee, A., et al. (2004) Vox Sang. 86, 141-7.

In vivo, the expression of VH4-34 gene derived antibodies is strictly regulated. Although 4-8% of human B cells express VH4-34 encoded antibody, serum levels of VH4-34 derived antibodies are negligible in normal adults. Stevenson F. K., et al., (1989) Br. J. Haematol. 72,9-15; Kraj P, et al., (1995) J. Immunol. 154,6406-6420. Increase in circulating VH4-34 derived antibodies is seen only in selective pathological conditions including viral infections (Epstein Barr (mononucleosis), human immunodeficiency virus and hepatitis C virus), Mycoplasma pneumoniae and certain autoimmune diseases. See also Bhat, N. M. et al., (2005) Human Antibodies 13, 63-68.

Accordingly, in one embodiment, a method is provided for reducing the amount of B cells or plasma cells producing pathologic antibodies in the body of a patient suffering from an autoimmune disease (i.e., reducing the numbers of B cells and plasma cells in the patient), comprising treating the patient with a therapeutically effective dose of an antibody having specific binding for an epitope present on germline antibodies. Preferably, the germline antibodies are selected from VH4-34, VH1-69, V71-2, V71-4, VH4-18, VH72-1, or V2-1 antibodies. In a particular embodiment, the antibody having specific binding for an epitope present on germline antibodies is 9G4, humanized 9G4, chimerized 9G4, or fragments or conjugates thereof. In additional embodiments, the antibody having specific binding for an epitope present on germline antibodies is 9G4, G6, 17.109, or LC1, humanized 9G4, G6, 17.109, or LC1, chimerized 9G4, G6, 17.109, or LC1, or fragments or conjugates thereof. The method can further comprise treating the patient with an additional pharmaceutically active agent, therapeutically effective treatment or other adjunct therapy. The additional pharmaceutically active agent can be a chemotherapeutic agent, complement activation inhibitor, antimetabolite (e.g., methotrexate), steroid, toleragen, anti-B cell agent, or anticoagulant (heparin, coumadin, antiplatelet agent such as acetylsalicylic acid, TICLID® (ticlopidine HCl), PLAVIX® (clopidogrel bisulfate)) or intravenous immunoglobulin. The therapeutically effective treatment includes plasmapheresis or leukopheresis.

In another embodiment, a method is provided for reducing the amount of VH4-34 antibody producing B cells or plasma cells in a patient suffering from an autoimmune disease, comprising administering a therapeutically effective amount of an antibody having specific binding for an epitope present on VH4-34 antibodies. Preferably, the antibody having specific binding for an epitope present on VH4-34 antibodies is 9G4, humanized or chimerized 9G4, or fragments or conjugates thereof.

In an additional embodiment, a method is provided for treating a patient suffering from cold agglutinin disease, comprising treating the patient with a therapeutically effective dose of an antibody having specific binding for an epitope present on germline antibodies. Preferably, the germline antibodies are selected from VH4-34, VH1-69, V71-2, V71-4, VH4-18, VH72-1, or V2-1 antibodies. In another preferred embodiment, the germline antibodies comprise Vkappa I, Vkappa III or Vkappa IV light chains, particularly VkappaIV light chains. In a particular embodiment, the germline antibodies are VH4-34 antibodies. In a preferred embodiment, the antibody having specific binding for an epitope present on germline antibodies is 9G4, humanized or chimerized 9G4, or fragments or conjugates thereof.

In an additional embodiment, a method is provided for treating a patient suffering from a B cell cancer expressing cell surface germline antibodies, comprising treating the patient with a therapeutically effective dose of an antibody having specific binding for an epitope present on germline antibodies. Preferably, the germline antibodies are selected from VH4-34, VH1-69, V71-2, V71-4, VH4-18, VH72-1, or V2-1 antibodies, and more preferably, the germline antibodies are VH4-34 or VH1-69 antibodies. The antibody having specific binding for an epitope present on germline antibodies is preferably 9G4, humanized or chimerized 9G4, or fragments or conjugates thereof. In additional embodiments, the antibody having specific binding for an epitope present on germline antibodies is 9G4, G6, 17.109, or LC1, humanized 9G4, G6, 17.109, or LC1, chimerized 9G4, G6, 17.109, or LC1, or fragments or conjugates thereof. The method can further comprise treating the patient with an additional pharmaceutically active agent selected from a chemotherapeutic agent, anti-B cell agent, cell growth regulator and/or inhibitor, immune modulator or combinations thereof. Preferably, the chemotherapeutic agent is asparaginase, epipodophyllotoxin, camptothecin, antibiotic, platinum coordination complex, alkylating agent, folic acid analog, pyrimidine analog, purine analog, topoisomerase inhibitor, or an agent that disrupts the cytoskeleton, or mixtures thereof. Preferred anti-B cell agents include antibodies or inhibitors of CD11a, CD19, CD20, CD21, CD22, CD25, CD34, CD37, CD38, CD40, CD45, CD52, CD80, CD 86, IL-4R, IL-6R, IL-8R, IL-13, IL-13R, α-4/β-1 integrin (VLA4), BLYS receptor, cell surface idiotypic Ig, CDIM, tumor necrosis factor (TNF), or combinations thereof. In a particular embodiment, the anti-B cell agent is an anti-CDIM antibody. The anti-CDIM antibody can be mAb 216, RT-2B, FS 12, A6(H4C5), Cal-4G, S20A2, FS 3, Gee, HT, Z2D2, or Y2K.

In alternative embodiments, methods are provided for purging the bone marrow of a patient suffering from autoimmune disease or B cell cancer prior to reimplantation of the bone marrow in the patient after myeloablative therapy, comprising treating the bone marrow of a patient ex vivo with a therapeutically effective amount of an antibody having specific binding for an epitope present on germline antibodies. The method can further comprise treating the bone marrow with an additional pharmaceutically active agent. Preferably, the antibody having specific binding for an epitope present on germline antibodies is 9G4, humanized or chimerized 9G4, or fragments or conjugates thereof. In additional embodiments, the antibody having specific binding for an epitope present on germline antibodies is 9G4, G6, 17.109, or LC1, humanized 9G4, G6, 17.109, or LC1, chimerized 9G4, G6, 17.109, or LC1, or fragments or conjugates thereof.

In another embodiment, a method is provided for purging the bone marrow of a patient suffering from autoimmune disease or B cell cancer prior to reimplantation of the bone marrow in the patient after myeloablative therapy, comprising treating the bone marrow of a patient ex vivo with a therapeutically effective amount of an antibody having specific binding for an epitope present on VH4-34 antibodies. The method can further comprise treating the bone marrow with an additional pharmaceutically active agent. The antibody having specific binding for an epitope present on VH4-34 antibodies is preferably 9G4, humanized or chimerized 9G4, or fragments or conjugates thereof.

In yet another embodiment, methods are provided for treating a patient suffering from autoimmune disease or a B cell cancer, comprising treating the patient with a therapeutically effective amount of an antibody having specific binding for an epitope present on germline antibodies, and further comprising treating the patient with a therapeutically effective amount of an anti-B cell agent. Preferably, the anti-B cell agent is an anti-CDIM antibody, selected from mAb 216, RT-2B, FS 12, A6(H4C5), Cal-4G, S20A2, FS 3, Gee, HT, Z2D2, or Y2K. In an additional aspect, the method includes providing sufficient time to allow the antibody having specific binding for an epitope present on germline antibodies to clear from the plasma of the patient prior to administering the anti-CDIM antibody. In another aspect, the method includes providing sufficient time to allow the anti-CDIM antibody to clear from the plasma of the patient prior to administering the antibody having specific binding for an epitope present on germline antibodies. A sufficient time generally is provided in 5 serum half-lives.

In an additional embodiment, a method for removing pathologic antibodies from the body of a patient suffering from autoimmune disease is provided, comprising contacting the blood or plasma of the patient with an immunoadsorbent having specific binding for an epitope present on germline antibodies. The method can further comprise readministering the contacted blood or plasma back to the patient, and/or administering additional normal blood or bone marrow or lymphoid tissue to the patient. Said contacting is typically effected using plasmapheresis or leukopheresis, and results in a reduction in the amount of germline antibodies present in the patient, and/or a reduction in the amount of cells (e.g., B cells or cancer cells of a B cell cancer) expressing germline antibodies in the patient. Preferably, the germline antibodies are selected from VH4-34, VH1-69, V71-2, V71-4, VH4-18, VH72-1, or V2-1 antibodies.

In a preferred embodiment, methods are provided for removing pathologic antibodies from the body of a patient suffering from autoimmune disease, comprising contacting the blood or plasma of the patient with an immunoadsorbent having specific binding for an epitope present on VH4-34 antibodies, wherein said contacting results in the reduction in the amount of VH4-34 antibodies present in the blood of the patient. Preferably, the immunoadsorbent comprises an antibody or antibody fragment having specific binding for an epitope present on VH4-34 antibodies, particularly 9G4, humanized 9G4, chimerized 9G4, or a fragment or conjugate thereof. The antibody fragment comprises a portion of the CDR imparting specific binding for VH4-34 antibodies. Preferably said contacting is effected using plasmapheresis or leukopheresis.

In an additional embodiment, a method is provided for reducing the amount of VH4-34 antibody producing B cells or plasma cells in a patient suffering from an autoimmune disease, comprising contacting the blood or plasma of the patient with an immunoadsorbent having specific binding for an epitope present on VH4-34 antibodies, wherein said contacting results in the reduction in the amount of VH4-34 antibody producing B cells present in the blood, lymphoid tissues or bone marrow of the patient.

In other embodiments, methods are provided for treating a patient suffering from a B cell cancer expressing cell surface VH4-34 immunoglobulin, comprising contacting the blood of the patient with an immunoadsorbent having specific binding for an epitope present on VH4-34 antibodies, wherein said contacting results in the reduction in the amount of VH4-34 antibody expressing B cells present in the blood, lymphoid tissues or bone marrow of the patient. B cell cancers include any acute leukemia, chronic leukemia, myeloma or lymphoma, and include aggressive, indolent or mantel cell lymphomas, and in particular, acute lymphocytic leukemia (ALL), non-Hodgkin's lymphoma (NHL), Hodgkin's Lymphoma, mantle cell lymphoma, Burkitt's lymphoma, B progenitor ALL, adult ALL, or chronic lymphocytic leukemia (CLL), and the like.

In any of the above embodiments, after contacting the blood of the patient with an immunoadsorbent, the patient can be further treated by administration of a therapeutic composition comprising cytolytic anti-germline or anti-VH4-34 antibody to further reduce the amount of circulating germline or VH4-34 antibody or cells producing or expressing germline or VH4-34 antibody.

II. Autoimmune Diseases

Autoimmune diseases are mediated by autoreactive antibodies, having binding specificity directed against self antigens. Patients suffering from autoimmune diseases typically have high serum titers of autoreactive antibodies, binding for example, to phospholipid, dsDNA, etc. Various auto-antibodies using the VH4-34 gene have been described, including the anti I/i cold agglutinins in autoimmune hemolytic anemia (Pascual, et al. (1991) J. Immunol. 146: 4385; Pascual et al., (1992) Arthr. Rheum. 35: 11; Silberstein, et al. (1991) Blood 78: 2372; Leoni, (1991) J. Biol. Chem. 266: 2836), anti-Rh monoclonal Abs (Borretzen, et al. (1995) Scan. J. Immunol. 42, 90), and polyreactive antibodies that bind DNA, lipid A, cardiolipin and rheumatoid factor Pascuel, et al. (1992) Arthritis Rheum. 35: 11).

Representative autoimmune diseases that can be treated using the methods and compositions described herein include cold agglutinin disease, systemic lupus erythematosis, rheumatoid arthritis, autoimmune lymphoproliferative disease, multiple sclerosis, psoriasis, and myasthenia gravis, but can also include Hashimoto's thyroiditis, lupus nephritis, dermatomyositis, Sjogren's syndrome, Sydenham's chorea, lupus nephritis, rheumatic fever, polyglandular syndromes, bullous pemphigoid, diabetes mellitus, Henoch-Schonlein purpura, post-streptococcal nephritis, erythema nodosum, Takayasu's arteritis, Addison's disease, Crohn's disease, Alzheimer's disease, sarcoidosis, ulcerative colitis, erythema multiforme, IgA nephropathy, polyarteritis nodosa, ankylosing spondylitis, Goodpasture's syndrome, thromboangitis ubiterans, primary biliary cirrhosis, thyrotoxicosis, scleroderma, chronic active hepatitis, polymyositis/dermatomyositis, polychondritis, pamphigus vulgaris, Wegener's granulomatosis, membranous nephropathy, amyotrophic lateral sclerosis, tabes dorsalis, giant cell arteritis/polymyalgia, pernicious anemia, rapidly progressive glomerulonephritis, fibrosing alveolitis, Class III autoimmune diseases such as immune-mediated thrombocytopenias, such as acute idiopathic thrombocytopenic purpura and chronic idiopathic thrombocytopenic purpura, and the like.

III. B Cell Cancers

B cell cancers include any cancer of B cell origin, and include all lymphoid cancers, particularly any acute leukemia of B cell origin. Lymphoid cancers include acute leukemias, such as acute lymphocytic leukemia (ALL), B progenitor ALL, adult ALL, as well as chronic leukemias, and lymphomas. Lymphomas include aggressive, indolent and mantel cell types. Particular examples of lymphoid cancer include without limitation acute lymphocytic leukemia (ALL), non-Hodgkin's lymphoma (NHL), Hodgkin's Lymphoma, mantle cell lymphoma, Burkitt's lymphoma, B progenitor ALL, adult ALL, or chronic lymphocytic leukemia (CLL), and the like.

IV. Additional Active Agents

Additional active agents include those utilized to treat autoimmune diseases or B cell cancers. Additional active agents useful in treating autoimmune diseases typically include chemotherapeutic agents, immune modulators such as NSAIDS (e.g., aspirin, naproxen); anti-inflammatory steroids (e.g., prednisolone, prednisone, or dexamethasone); antiproliferative/antimetabolic agents (e.g., azathioprine, chlorambucol, cyclophosphamide, leflunomide, mycophenolate mofetil, methotrexate hydrate, rapamycin, thalidomide); cyclosporine A; antimalarial agents (e.g., hydrochloroquine); tacrolimus (FK 506) and ascomycin. Immune modulators can also include cytokines such as interleukins (e.g., IL-21). Additional active agents can include treatment with anti-B cell agents such as antibodies or inhibitors of CD11a, CD19, CD20, CD21, CD22, CD25, CD34, CD37, CD38, CD40, CD45, CD52, CD80, CD 86, IL-4R, IL-6R, IL-8R, IL-13, IL-13R, α-4/β-1 integrin (VLA4), BLYS receptor, cell surface idiotypic Ig, tumor necrosis factor (TNF), or combinations thereof, without limitation. Anti-B cell agents can act by cytotoxic mechanisms or immunomodulatory mechanisms. For example, the antibody to CD11a can be, for example, efalizumab (RAPTIVA). The antibody to CD20 can be rituximab (RITUXAN). The antibody to CD22 can be, for example, epratuzumab. The antibody to CD25 can be, for example, daclizumab (ZENAPAX) or basiliximab (SIMULECT). Antibodies to CD52 include, e.g., CAMPATH. Antibodies to α-4/β-1 integrin (VLA4) include, e.g., natalizumab. Antibodies to TNF include, for example, infliximab (REMICADE). Preferred anti-B cell agents include antibodies to CD 20 (e.g., rituximab), CD22, CD23, CD 40, CD40 ligand, CDIM epitope, anti-idiotype antibodies, and the like. Anti-CDIM binding agents preferably comprise an antibody selected from mAb 216, RT-2B, FS 12, A6(H4C5), Cal-4G, S20A2, FS 3, Gee, HT, Z2D2, or Y2K.

An additional class of immune modulators includes toleragens such as abetimus sodium (LJP-394), LJP 993 and LJP 1082. Agents that are also useful include anti-T cell agents, e.g., agents that block T cell mediated disease (co-stimulatory pathway inhibitors such as anti-CTLA-4, anti-CD40 ligand, anti-alpha-4-integrins such as anti-VLA-4 (natalizumab or Tysabri) and abatacept (CTLA4-Ig, BMS-188667), adhesion molecule inhibitors (anti-ICAM 1 anti-CD11b/CD18).

Additional active agents include intravenous immunoglobulin, particularly post-plasmapheresis, and complement activation inhibitors (e.g., the anti-C5 agents pexelizumab or eculizumab, soluble CR1). Mixtures of any of the agents discussed above or combinations of these treatments can also be utilized.

In addition, active agents useful for treating B cell cancers include chemotherapeutic agents, radioactive isotopes, cytotoxic antibodies, immunoconjugates, ligand conjugates, immunosuppressants, cell growth regulators and/or inhibitors, toxins, or mixtures thereof.

Chemotherapeutic Agents:

The chemotherapeutic agents that can be used in the formulations and methods of the invention include taxanes, colchicine, vinca alkaloids, epipodophyllotoxins, camptothecins, antibiotics, platinum coordination complexes, alkylating agents, folic acid analogs, pyrimidine analogs, purine analogs or topoisomerase inhibitors. A preferred topoisomerase inhibitor is an epipodophyllotoxin. Preferred pyrimidine analogs include capecitabine, 5-fluoruracil, 5-fluorodeoxyuridine, 5-fluorodeoxyuridine monophosphate, cytosine arabinoside, 5-azacytidine, or 2′,2′-difluorodeoxycytidine. Preferred purine analogs include mercaptopurine, azathioprene, thioguanine, pentostatin, erythrohydroxynonyladenine, cladribine, vidarabine, and fludarabine phosphate. Folic acid analogs include methotrexate, raltitrexed, lometrexol, permefrexed, edatrexate, and pemetrexed. A preferred epipodophyllotoxin is etoposide or teniposide. A preferred camptothecin is irinotocan, topotecan, or camptothecan. Preferably, the antibiotic is dactinomycin, daunorubicin (daunomycin, daunoxome), doxorubicin, idarubicin, epirubicin, valrubucin, mitoxanthrone, bleomycin, or mitomycin. A preferred platinum coordination complex is cisplatin, carboplatin, or oxaliplatin. Preferably, the alkylating agent is mechlorethamine, cyclophosphamide, ifosfamide, melphalan, dacarbazine, temozolomide, thiotepa, hexamethylmelamine, streptozocin, carmustine, busulfan, altretamine or chlorambucil.

Additional examples of chemotherapeutic agents can include alkylating agents such as thiotepa and cyclosphosphamide (CYTOXAN™);

alkyl sulfonates such as busulfan, improsulfan and piposulfan;

aziridines such as benzodopa, carboquone, meturedopa, and uredopa;

ethylenimines and methylamelamines including altretamine, triethylenemelamine, trietylenephosphoramide, triethylenethiophosphoramide and trimethylolomelamine;

acetogenins (especially bullatacin and bullatacinone);

camptothecins (including the synthetic analogue topotecan);

bryostatin; callystatin; CC-1065 (including its adozelesin, carzelesin and bizelesin synthetic analogues);

cryptophycins (particularly cryptophycin 1 and cryptophycin 8);

dolastatin; duocarmycin (including the synthetic analogues, KW-2189 and CBI-TMI);

eleutherobin; pancratistatin; sarcodictyin; spongistatin;

nitrogen mustards such as chlorambucil, chlomaphazine, cholophosphamide, estramustine, ifosfamide, mechlorethamine, mechlorethamine oxide hydrochloride, melphalan, novembichin, phenesterine, prednimustine, trofosfamide, uracil mustard;

nitrosureas such as carmustine, chlorozotocin, fotemustine, lomustine, nimustine, ranimustine;

antibiotics such as the enediyne antibiotics (e.g. calicheamicin, especially calicheamicin gamma1I and calicheamicin phiI1, see, e.g., Agnew (1994) Chem. Intl. Ed. Engl., 33:183-186; dynemicin, including dynemicin A; bisphosphonates, such as clodronate; esperamicin; as well as neocarzinostatin chromophore and related chromoprotein enediyne antibiotic chromomophores), aclacinomysins, actinomycin, authramycin, azaserine, bleomycins, cactinomycin, carabicin, carminomycin, carzinophilin, chromomycins, dactinomycin, daunorubicin, detorubicin, 6-diazo-5-oxo-L-norleucine, doxorubicin (Adriamycin™) (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin;

anti-metabolites such as methotrexate and 5-fluorouracil (5-FU);

folic acid analogues such as denopterin, methotrexate, pteropterin, trimetrexate;

folic acid replenisher such as folinic acid;

purine analogs such as fludarabine, 6-mercaptopurine, thiamiprine, thioguanine;

pyrimidine analogs such as ancitabine, azacitidine, 6-azauridine, carmofur, cytarabine, dideoxyuridine, doxifluridine, enocitabine, floxuridine;

androgens such as calusterone, dromostanolone propionate, epitiostanol, mepitiostane, testolactone;

anti-adrenals such as aminoglutethimide, mitotane, trilostane;

aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone; etoglucid; gallium nitrate; hydroxyurea; lentinan; lonidamine; maytansinoids such as maytansine and ansamitocins; mitoguazone; mitoxantrone; mopidamol; nitracrine; pentostatin; phenamet; pirarubicin; losoxantrone; podophyllinic acid; 2-ethylhydrazide; procarbazine; PSK®; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; cytosine, arabinoside (“Ara-C”);

cyclophosphamide; thiotepa; taxoids, e.g. paclitaxel (TAXOL®, Bristol-Myers Squibb Oncology, Princeton, N.J.) and doxetaxel (TAXOTERE®, Rhone-Poulenc Rorer, Antony, France); chlorambucil; gemcitabine (Gemzar™); 6-thioguanine; mercaptopurine; methotrexate;

platinum analogs such as cisplatin and carboplatin;

vinblastine, vincristine; vinorelbine (Navelbine™);

etoposide (VP-16); ifosfamide; mitoxantrone; novantrone; teniposide; edatrexate; daunomycin; aminopterin; xeloda; ibandronate; CPT-11;

topoisomerase inhibitor RFS 2000; difluoromethylomithine (DMFO);

retinoids such as retinoic acid; capecitabine; and pharmaceutically acceptable salts, acids or derivatives of any of the above.

Additional preferred chemotherapeutic agents include those used in combination therapies, for example, CHOP, and so forth. In particular embodiments, such combination therapies can be used with the anti-CDIM binding antibodies, or in combination with additional cytotoxic antibodies, in particular anti-CD22, anti-CD52 and anti-CD20 antibodies.

Particularly preferred are agents that arrest the B cell in its cell cycle, such as agents that interfere with the polymerization or depolymerization of microtubules. Exemplary agents include colchicine, the vinca alkaloids, such as vincristine, vinblastine, vindesine, or vinorelbine, and taxanes, such as taxol, paclitaxel, and docetaxel. Additional preferred agents are anti-actin agents. In a preferred embodiment, the anti-actin agent is j asplakinolide or cytochalasin, which can be used more preferably in an ex vivo method, such as a method of purging bone marrow of malignant cells. Mixtures of any of the above agents can also be used, such as CHOP, CAMP, DHAP, EPIC, and the like, as discussed in U.S. patent application Ser. No. 2004/0136951, incorporated by reference herein.

Conjugates

The term “conjugate” refers to coupling of active agents, which can be covalent or noncovalently associated, and includes immunoconjugates or conjugates of other ligands (such as anti-B cell agents that bind to B cell associated surface molecules). Immunoconjugates are conjugates of antibodies to active agents, and include therapeutic compositions such as conjugates of toxins, radioisotopes, or compositions useful in monitoring the efficacy of treatment, such as conjugates comprising indicator molecules such as colloidal beads, fluorescent dyes, radioisotopes, and the like. Immunoconjugates can be prepared by numerous methods known in the art, such as chemical derivatization of the antibody to provide reactive crosslinking groups, which can be labile or non-labile. Labile reactive groups provide for the release of the cytotoxic agent or growth regulator from the antibody. Non-labile crosslinking is also useful. The linkage of the desired agent to the Ig molecule may be achieved by a variety of means known to the art including conventional coupling techniques (e.g., coupling with dehydrating agents such as dicyclohexylcarbodiimide (DCCI), ECDI and the like), the use of linkers capable of coupling through sulfhydryl groups, amino groups or carboxyl groups (available from Pierce Chemical Co., Rockford, Ill.), by reductive amination.

In one method, an antibody conjugate, or immunoconjugate, can be prepared by first modifying the antibody with a cross-linking reagent such as N-succinimidyl pyridyldithiopropionate (SPDP) to introduce dithiopyridyl groups into the antibody (Carlsson et al. (1978) Biochem. J. 173, 723-737; U.S. Pat. No. 5,208,020). In a second step, an agent having a thiol group, is added to the modified antibody, resulting in the displacement of the thiopyridyl groups in the modified antibodies, and the production of disulfide-linked agent-antibody conjugate. A procedure to prepare maytansinoid-antibody conjugates is described in U.S. Pat. No. 5,208,020.

Toxins can be administered as immunoconjugates, ligand conjugates, or co-administered with an antibody. Toxins include, without limitation, Pseudomonas exotoxin A, ricin, diphtheria toxin, momordin, pokeweed antiviral protein, Staphylococcal enterotoxin A, gelonin, maytansinoids (e.g., as described in U.S. Pat. Nos. 6,441,163), or the like.

Radioisotopes

The isotopes used to produce therapeutically useful immuno- or ligand conjugates typically produce high energy α-, γ- or β-particles which have a therapeutically effective path length. Such radionuclides kill cells to which they are in close proximity, for example neoplastic cells to which the conjugate is bound. The advantage of targeted delivery is that the radioactively labeled antibody or ligand generally has little or no effect on cells not in the immediate proximity of the targeted cell. Isotopes useful in assays or kits for monitoring therapeutic efficacy typically produce energies that can be detected using conventional laboratory equipment and include the commonly used radioisotopes ³H, ¹⁴C and ³²P, and the like.

Antibodies or ligands (e.g., anti-B cell agents) may be directly labeled (such as through iodination or phosphorylation) or can be conjugated using of a chelating agent. In either method, the antibody or ligand is labeled with at least one radionuclide. Particularly preferred chelating agents comprise l-isothiocyamatobenzyl-3-methyldiothelene triaminepentaacetic acid (“MX-DTPA”) and cyclohexyl diethylenetriamine pentaacetic acid (“CHX-DTPA”) derivatives. Other chelating agents comprise P-DOTA and EDTA derivatives. Particularly preferred radionuclides for indirect labeling include ¹¹¹In and ⁹⁰Y.

The radioactive isotope can be attached to specific sites on the antibody or ligand, such as the N-linked sugar resides present only on the Fc portion of the antibody. Technetium-99m labeled antibodies or ligands may be prepared by ligand exchange processes or by batch labeling processes. For example, the antibody can be labeled by reducing pertechnate (TcO₄) with stannous ion solution, chelating the reduced technetium onto a Sephadex column and applying the antibody to this column. Batch labeling techniques include, for example, incubating pertechnate, a reducing agent such as SnCl₂, a buffer solution such as a sodium-potassium phthalate-solution, and the antibody. Preferred radionuclides for labeling are well known in the art. An exemplary radionuclide for labeling is ¹³¹I covalently attached via tyrosine residues. Radioactively labeled antibodies according to the invention can be prepared with radioactive sodium or potassium iodide and a chemical oxidizing agent, such as sodium hypochlorite, chloramine T or the like, or an enzymatic oxidizing agent, such as lactoperoxidase, glucose oxidase and glucose.

Patents relating to chelators and chelator conjugates are known in the art. For example, U.S. Pat. No. 4,831,175 to Gansow is directed to polysubstituted diethylenetriaminepentaacetic acid chelate and protein conjugates containing the same and methods for their preparation. U.S. Pat. Nos. 5,099,069, 5,246,692, 5,286,850, 5,434,287 and 5,124,471 all to Gansow also relate to polysubstituted DTPA chelates. These patents are incorporated herein by reference in their entireties. Other examples of compatible metal chelators are ethylenediaminetetraacetic acid (EDTA), diethylenetriaminepentaacetic acid (DPTA), 1,4,8,11-tetraazatetradecane, 1,4,8,11 tetraazatetradecane-1,4,8,11-tetraacetic acid, 1-oxa-4,7,12,15-tetraazaheptadecane, 4,7,12,15-tetraacetic acid, or the like. Cyclohexyl-DTPA or CHX-DTPA is particularly preferred. Still other compatible chelators, including those yet to be discovered, may easily be discerned by a skilled artisan and are clearly within the scope of the present invention. Additional chelators include the specific bifunctional chelators described in U.S. Pat. Nos. 6,682,734, 6,399,061 and 5,843,439, and are preferably selected to provide high affinity for trivalent metals, exhibit increased tumor-to-non-tumor ratios and decreased bone uptake as well as greater in vivo retention of radionuclide at target sites, i.e., B-cell lymphoma tumor sites. However, other bifunctional chelators that may or may not possess all of these characteristics are known in the art and may also be beneficial in tumor therapy.

Modified antibodies can also be conjugated to radioactive labels for diagnostic as well as therapeutic purposes (e.g., for use in assays or kits). Radiolabeled therapeutic conjugates for diagnostic “imaging” of tumors can also be utilized before administration of antibody and cytotoxic agent to a patient. For example, the monoclonal antibody binding the human CD20 antigen known as C2B8 can be radiolabeled with ¹¹¹In using a bifunctional chelator, such as MX-DTPA (diethylenetriaminepentaacetic acid), which comprises a 1:1 mixture of 1-isothiocyanatobenzyl-3-methyl-DTPA and 1-methyl-3-isothiocyanatobenzyl-DTPA. ¹¹¹In is a preferred diagnostic radioactive isotope since between about 1 and about 10 mCi can be safely administered without detectable toxicity, and the imaging data is an indicator of subsequent ⁹⁰Y-labeled antibody distribution. A typical dose of ¹¹¹In-labeled antibody of 5 mCi for imaging studies is used, and optimal imaging can be determined at various times after administration of the labeled antibody or ligand, typically three to six days after administration. See, for example, Murray, J. (1985) Nuc. Med. 26, 3328 and Carraguillo et al., (1985) J. Nuc. Med. 26, 67.

A variety of radioactive isotopes can be utilized and one skilled in the art can readily determine which radioactive isotope is most appropriate under various conditions. For example, ¹³¹I is frequently utilized for targeted immunotherapy. However, the clinical usefulness of ¹³¹I can be limited by its short half life (8 days), the potential for dehalogenation of iodinated antibody both in the blood and at tumor or sites, and its high energy γ emission which may not provide sufficiently localized dose deposition in tumor, depending on tumor size, as desired. With the advent of additional chelating agents, additional opportunities are provided for attaching metal chelating groups to proteins and utilizing other radionuclides such as ¹¹¹In and ⁹⁰Y. ⁹⁰Y provides several benefits for utilization in radioimmunotherapeutic applications. For example, the longer useful half life of 64 hours for ⁹⁰Y is sufficiently long to allow antibody accumulation by tumor cells and, unlike ¹³¹I, ⁹⁰Y is a pure beta emitter of high energy with no accompanying gamma radiation in its decay, having a range in tissue of 100 to 1,000 cell diameters. Furthermore, the minimal amount of penetrating radiation allows for outpatient administration of ⁹⁰Y-labeled antibodies. Additionally, internalization of labeled antibody is not required for cell killing, and the ionizing radiation should be lethal for adjacent tumor cells lacking the target antigen.

V. Immunoadsorption and Therapeutic Plasmapheresis and Leukopheresis

Plasmapheresis has been utilized for treatment of patients suffering from autoimmune diseases, with apparent efficacy due to removal of antibodies, immune complexes, proinflammatory agents and soluble adhesion molecules. Immunoadsorption in conjunction with plasmapheresis has been utilized for the removal of IgG using Staphylococcal protein A or anti-human Ig antibodies. See for example, Graninger, M. et al., (2002) Acta Med. Austriaca 29, 26-29 (use of polyclonal sheep anti-human Ig conjugated column “Ig-Therasorb/Pt” (Therasorb, Munchen, Germany) to remove human immunoglobulins from plasma). Leukopheresis can be utilized to remove cells bearing cell surface antibodies using similar methods.

Plasmapheresis and imunoadsorption procedures and devices are known in the art, and typically involve the separation of plasma from cellular blood components using centrifugation. Instruments can be calibrated to perform plasmapheresis, plateletpheresis (collection of donor platelets for patient use), erythrocytopheresis (used for treatment of sickle cell anemia), or leukopheresis (collection of donor stem cells for transplantation; removal of white blood cells for therapeutic purposes). Differential cell density gradients allow centrifugal separators to apherese by continuous or discontinuous methods. Hollow fiber or rotating cylinder membranes can also be used to effect separation. Membranes can be used with a dialyzer or a centrifugation device to separate blood constituents using a filtration process, allowing lower molecular weight components to pass through the membrane while retaining higher molecular weight components. A typical membrane comprises cellulose acetate, although a variety of materials can be designed to selectively retain specific plasma components by cryoprecipitation (removal of cryoglobulins) or affinity adsorption (e.g., removal of IgG-class antibodies by adsorption to Staphylococcus protein A). Membranes can be utilized singly or multiply so that the first membrane separates plasma from cellular components and the second selectively removes specific plasma components.

The term immunoadsorbent (or immnosorbent) is used in its broadest sense to refer to matrices capable of immunospecifically binding to a desired epitope comprising filters, membranes, particles, beads, and the like, as well as monolithic materials. Immunoadsorbents derivatized with monoclonal antibodies provide a means for the highly specific removal of plasma proteins. Coupling techniques well known in the art can also be utilized to prepare immunoadsorbents having a desired specific binding. Immunoadsorbents can be utilized to remove circulating antibodies from the blood or plasma of a patient or to remove cells bearing the target antibodies on their cell surfaces from the blood or bone marrow or lymphoid tissues. Immunoadsorbent materials such as dextran sulfate columns have been shown to lower circulating levels of antiDNA and antiphospholipid antibodies and circulating immune complexes. Columns containing polyvinyl gels to which phenylalanine or tryptophan have been added were reported to eliminate antiDNA and antiphospholipid antibodies, immune complexes, and rheumatoid factor (RF) by hydrophobic interactions. Staphylococcus protein A columns were shown to bind IgG subclasses 1, 2, and 4 and can be used before transplantation or for patients with hemophilia in addition to those with autoimmune diseases. Antihuman IgG columns (with specificities for the heavy and light chains) remove virtually all IgG antibodies and substantially reduce IgM and IgA antibodies.

Preferably, the immunoadsorbent comprises an antibody or portion of an antibody having specific binding for an epitope present on germline antibodies. A preferred immunoadsorbent comprises an anti-germline antibody or portions of anti-germline antibody able to specifically bind to germline antibodies, preferably VH4-34, VH1-69, V71-2, V71-4, VH4-18, VH72-1, or V2-1 antibodies. In certain embodiments, the immunoadsorbent comprises a ligand possessing the same binding characteristics as the anti-germline antibody able to specifically bind the germline antibodies. In a particular embodiment, the immunoadsorbent comprises anti-germline antibodies having specific binding for VH4-34 antibodies, preferably 9G4 or LC1 or portions thereof. In another embodiment, the immunoadsorbent comprises a ligand capable of binding VH4-34 antibodies, or the portion of VH4-34 antibodies comprising the amino acid sequence for framework region 1 and/or the amino acid sequence AVY.

Methods described herein comprise contacting the blood or plasma of the patient with an immunoadsorbent having specific binding for an epitope present on germline antibodies or cells. Plasmapheresis provides a convenient method for contacting the blood or plasma with an immunoadsorbent to remove these pathologic antibodies or cells. However, any other method providing specific binding and removal of antibodies can be utilized.

VI. Methods for Monitoring the Efficacy of a Therapeutic Treatment

In one aspect, methods are provided for monitoring the efficacy of treatment provided to a patient suffering from an autoimmune disease such as SLE or lupus nephritis, for example. The method generally comprises obtaining a sample of blood or bone marrow or lymphoid tissue from the patient, contacting the sample with an amount of anti-germline antibody sufficient to bind to germline antibodies present in the sample of blood or bone marrow or lymphoid tissue, determining the amount of anti-germline antibody bound in the sample of blood or bone marrow or lymphoid tissue, and correlating the amount of anti-germline antibody bound with the reduced amount or continued presence of germline antibody, and therefore the efficacy or lack of efficacy of treatment to reduce the number of germline antibody producing B cells or the amount of germline antibody in the sample of blood or bone marrow or lymphoid tissue obtained from the patient at a time period prior to initiation of the therapeutic treatment. Preferably the anti-germline antibody is associated with a substrate for performing an ELISA or radioimmunoassay. The anti-germline antibody can also be utilized in flow cytometry to determine numbers of cells present expressing cell surface germline antibody. Therapeutic treatments include plasmapheresis, leukopheresis, or treatment with an anti-germline antibody or additional pharmaceutically active agent comprising an anti-B cell agent, chemotherapeutic agent, toleragen, complement activation inhibitor, antimetabolite, steroid, anticoagulant or intravenous immunoglobulin, or combinations thereof.

In additional aspects, methods for monitoring the efficacy of a therapeutic treatment in a patient suffering from an autoimmune disease are provided, comprising obtaining a sample of blood or bone marrow or lymphoid tissue from the patient, contacting said sample with an amount of anti-VH4-34 antibody sufficient to bind to VH4-34 antibodies present in the sample of blood or bone marrow or lymphoid tissue, determining the amount of anti-VH4-34 antibody bound in the sample of blood or bone marrow or lymphoid tissue, and correlating the amount of anti-VH4-34 antibody bound with the efficacy of treatment to reduce the number of VH4-34 antibody producing B cells or the amount of VH4-34 antibody in the sample of blood or bone marrow or lymphoid tissue obtained from the patient at a time period prior to initiation of the therapeutic treatment. In a preferred embodiment, the anti-VH4-34 antibody is associated with a substrate for performing an ELISA or radioimmunoassay. In another preferred embodiment, the anti-VH4-34 antibody is utilized in flow cytometry. Suitable therapeutic treatments include plasmapheresis, leukopheresis, or treatment with an additional pharmaceutically active agent comprising an anti-B cell agent, chemotherapeutic agent, toleragen, complement activation inhibitor, antimetabolite, steroid, anticoagulant or intravenous immunoglobulin.

A sample of serum from a patient is obtained and treated according to the following steps of (a) combining the sample with an anti-germline antibody, preferably a VH4-34 binding antibody (e.g., the rat monoclonal antibody 9G4) wherein the sample is prepared by diluting serum with aqueous buffer at a volume ratio of sample to buffer of up to 1:1000; (b) performing a binding assay to determine the proportion of anti-germline antibody bound in the sample (e.g., the amount of 9G4 bound to VH4-34 antibody in the sample); and (c) comparing the result of step (b) to a control (e. g., results from group serum samples obtained from patients who are not suffering from autoimmune disease), to determine if said proportion is indicative of a reduction in the levels of circulating germline antibodies in the patient. In one embodiment, the volume ratio of sample to buffer is up to 1:100. The sample can be adjusted by dilution with aqueous buffer to yield a total Ig levels within any selected range, preferably within the range for normal serum. The method can include the step of determining the sample to be substantially free from rheumatoid factor antibody, in order to reduce false positive results from patients having rheumatoid arthritis.

The monitoring of treatment efficacy can also include patient assessment measures that are well known in the art of medical diagnosis and practice. For example, the monitoring of treatment efficacy can include monitoring disease progression or amelioration of symptoms using various well known clinical activity scales. For assessing SLE, clinical activity scales include the Systemic Lupus Activity Measure (“SLAM”), the Systemic Lupus Erythematosis Disease Activity Index (“SLEDAI”), and the British Isles Lupus Assessment Group (“BILAG”). For assessing rheumatoid arthritis, the American College of Rheumatology Response Criteria is commonly utilized (ACR20, ACR 50 and ACR 70 indicating 20%, 50% and 70% improvement, respectively). For Crohn's Disease, the Crohn's Disease Activity Index (CDAI) can be utilized. Results from the above described methods for assaying pathologic antibodies and pathologic antibody producing B-cells can be validated by correlation with these clinical activity scales.

In another aspect, methods are provided for monitoring the efficacy of treatment provided to a patient suffering from a B cell cancer such as ALL or CLL, for example. The method generally comprises obtaining a sample of blood or bone marrow or lymphoid tissue from the patient, contacting the sample with an amount of anti-germline antibody sufficient to bind to germline antibodies present in the sample of blood or bone marrow or lymphoid tissue, determining the amount of anti-germline antibody bound in the sample of blood or bone marrow or lymphoid tissue, and correlating the amount of anti-germline antibody bound with the reduced amount or continued presence of germline antibody, and therefore the efficacy or lack of efficacy of treatment to reduce the number of gerinline antibody producing B cells or the amount of germline antibody in the sample of blood or bone marrow or lymphoid tissue obtained from the patient at a time period prior to initiation of the therapeutic treatment.

ELISA

In another embodiment of the method, in step (a) the sample is subjected to an enzyme linked immunosorbent assay (“ELISA”) using a labeled reagent and a reagent bound to an insoluble phase material, wherein the labeled reagent is enzyme-labeled anti-germline antibody (such as the VH4-34 binding antibody, 9G4), the reagent bound to the insoluble phase material is germline antibody (e.g., a VH4-34 antibody), and said step (b) includes determining the enzyme-labeled anti-germline antibody which is bound to the insoluble phase material. In still another embodiment of the method, in step (a) the sample is subjected to an ELISA immunoassay using a labeled reagent and a reagent bound to an insoluble phase material, wherein the labeled reagent is enzyme-labeled germline antibody (e.g., VH4-34 antibody), the reagent bound to the insoluble phase material is anti-germline antibody (e.g., anti-VH4-34 antibody), and said step (b) includes determining the enzyme labeled VH4-34 antibody bound to the insoluble phase material. In a further embodiment of the method, in step (a) the sample is subjected to an ELISA immunoassay using a first reagent, and a second reagent bound to an insoluble phase material, wherein the first reagent is an anti-germline antibody (such as 9G4), the reagent bound to the insoluble phase material is VH4-34 antibody, and said step (b) includes determining the anti-germline antibody which is bound to the insoluble phase material by contacting the insoluble phase material with a labeled antibody which binds with the anti-germline antibody.

In a particular embodiment, total VH4-34 Igs in serum can be detected by an inhibition ELISA, for example as described in van Vollenhoven, R. F., et al., (1999) “VH4-34 encoded antibodies in SLE: a specific diagnostic marker that correlates with clinical disease characteristics,” J. Rheumatol. 26, 1727-1733. Briefly, plates are coated with purified VH4-34 IgM. Serum samples are incubated with 9G4 for 15 minutes at RT before transfer to VH4-34 IgM coated 96-well plates. The amount of 9G4 bound to the coated VH4-34 IgM is detected using peroxidase-labeled anti-rat IgG (Caltag, South San Francisco, Calif.). The VH4-34 encoded Abs in serum sample compete with coated VH4-34 IgM for 9G4 binding, leading to a range in color development depending upon the amount of VH4-34 Ig in patient serum.

VH4-34 encoded IgM and VH4-34 encoded IgG in serum can be detected, for example, as described in N. M. Bhat, N. M., et al., (2002) “V4-34 Encoded Antibody in SLE: Effect Of Isotype,” J. Rheumatol. 29,2114-2121. Briefly, plates are coated with purified 9G4 and detected with peroxidase labeled anti-human IgG or IgM. This assay provides relative amounts of each isotype of VH4-34 antibody in each serum specimen.

In another aspect, methods for monitoring the reduction in pathologic antibodies from a sample of serum from a patient are disclosed, with reduced false positive cross-reactions from rheumatoid arthritis, including the steps of (a) combining the sample with a binding fragment (e.g., Fab′, Fab or Fv, etc.) of 9G4 monoclonal antibody, wherein the sample is prepared by diluting serum with aqueous buffer at a volume ratio of sample to buffer of up to 1:1000; (b) determining the proportion of the binding fragment of 9G4 monoclonal antibody which has bound to VH4-34 antibody in the sample; (c) comparing the result of step (b) to a standard to determine if said proportion is sufficient to monitor the efficacy of treatment of SLE in the patient. In one embodiment, the volume ratio of sample to dilution buffer is up to 1:100. In a preferred embodiment, the sample is adjusted by dilution with aqueous buffer to yield a total IgG level within a selected range, preferably within the range for normal serum.

Flow Cytometry

Methods for performing flow cytometry to determine the numbers of receptors on cell surfaces are well known. Suitable flow cytometers are manufactured by Beckman Coulter Inc. (Fullerton, Calif.) or B D Biosciences (San Jose, Calif.). In general the flow cytometer sends cells in a single stream past a laser that excites a fluorophore present on an antibody or other labeled ligand bound to a cell surface antigen on the cell. The cells are incubated with fluorescently labeled probes (such as antibodies or dyes) that recognize molecules of interest such as cell surface antigens prior to being loaded into the cytometer. A set of optics focuses the lasers on passing labeled cells. When the excited fluorophores on the cells emit light, another set of optics collects the emitted light and sends it to filters that separate the emission spectra. Different wavelengths of light are detected by different detectors, and provide a record of how much light was emitted by each cell, a function of how much label was bound to each cell. The data is typically expressed in the form of a histogram, which can be interpreted to determine what percentage of the analyzed cell sample expresses a particular level of the ligand (e.g. antibody) of interest. The data can also be analyzed based on light scatter to provide size and shape information about the cells. Cells that exhibit binding of a certain amount of the label or are a certain shape or size can be analyzed separately using sorters that have the added function of sorting but can also be used just for their analysis capabilities.

Kits

In a further aspect, the a kit is provided for monitoring the therapeutic response in a patient in need thereof to administration of a treatment for autoimmune disease or B cell caner, comprising an amount of anti-germline antibody effective to bind to germline antibodies present in a sample of blood or bone marrow or lymphoid tissue from a patient suffering from said autoimmune disease or B cell cancer. In a preferred embodiment, the kit comprises an amount of anti-VH4-34 antibody effective to bind to VH4-34 antibodies present in a sample of blood or bone marrow or lymphoid tissue from a patient suffering from said autoimmune disease or B cell cancer. The kit can also comprise instructions for performing and interpreting the binding results obtained. The treatment can be any treatment for autoimmune disease, including administration of plasmapheresis, leukopheresis or administration of an antibody having specific binding for an epitope present on germline antibodies, including VH4-34 antibodies, or other pharmaceutically active agent.

Preferred kits include calibrated reagents comprising a binding fragment of anti-germline antibody, particularly 9G4 monoclonal antibody and germline antibody, particularly, VH4-34 reagent antibody. In one embodiment, the kit includes a labeled fragment of 9G4 monoclonal antibody. In a preferred embodiment, the kit includes VH4-34 antibody bound to an insoluble phase material (e.g., a substrate such as an ELISA plate). Additional reagents can also be included in the kits as desired, for example, control antibodies, secondary antibodies, supplies for ELISA assays, radioimmunoassay, instructions, or the like.

VII. Compositions

Antibodies and additional active agents can be formulated using any methods and pharmaceutically acceptable excipients known in the art. Typically, antibodies are provided in saline, with optional excipients and stabilizers. Additional active agents can vary widely in formulation methods and excipients, and this information is available for example, in Remington's Pharmaceutical Sciences (Arthur Osol, Editor).

The antibodies and methods described herein are also provided for use of an anti-germline antibody in the manufacture of a medicament for the treatment of autoimmune disease or B cell cancer. Preferably, the anti-germline antibody has specific binding activity for a VH4-34 antibody. In a further aspect, the composition consists essentially of a binding fragment of 9G4 monoclonal antibody. The binding fragment of 9G4 monoclonal antibody can be labeled, e.g. enzyme-labeled.

The antibodies can also be utilized in the preparation of immunoadsorbents for use in plasmapheresis and leukopheresis, wherein the immunoadsorbent comprises an anti-germline antibody or fragment thereof associated with a substrate (e.g., a sorbent) suitable for use in a plasmapheresis or leukopheresis apparatus. Preferably, the anti-germline antibody is selected from an antibody having specific binding for VH4-34, VH1-69, V71-2, V71-4, VH4-18, VH72-1, or V2-1 antibodies, and more preferably, an anti-VH4-34 antibody such as 9G4, humanized or chimerized 9G4, or fragments or conjugates thereof. In additional embodiments, the anti-germline antibody is 9G4, G6, 17.109, or LC1, humanized 9G4, G6, 17.109, or LC1, chimerized 9G4, G6, 17.109, or LC1, or fragments or conjugates thereof.

VIII. Modes of Administration

It is contemplated that the methods and compositions described herein can be used in in vivo, ex vivo and in vitro applications. For in vivo applications, the therapeutic compositions of the invention can be administered to the patient by a variety of different means. The means of administration will vary depending upon the intended application. As one skilled in the art would recognize, administration of the therapeutic compositions can be carried out in various fashions, and more typically by parenteral injection into a body cavity or vessel, e.g., intraperitoneal, intravenous, intralymphatic, intratumoral, intramuscular, interstitial, intraarterial, subcutaneous, intralesional, intraocular, intrasynovial, intraarticular. However, other methods of administration can be utilized for particular purposes, for example, via topical administration, including, but not limited to, dermal, ocular and rectal; transdermal, via passive or active means, e.g., using a patch, a carrier, or iontophoresis; transmucosal, e.g., sublingual, buccal, rectal, vaginal, or transurethral; oral, e.g., gastric or duodenal; via inhalation, e.g., pulmonary or nasal inhalation, using e.g., a nebulizer.

The antibody formulations can be administered by a relatively slow, sustained delivery from a drug receptacle, such as by subcutaneous administration into a pocket created by pinching or drawing the skin up and away from underlying tissue. A subcutaneous bolus can be administered, where the bolus drug delivery is preferably less than approximately 15 minutes, more preferably less than 5 minutes, and most preferably less than 60 seconds. A subcutaneous infusion of a relatively slow, sustained delivery from a drug receptacle can be performed over a period of time including, but not limited to, 30 minutes or less, or 90 minutes or less. Optionally, the infusion may be made by subcutaneous implantation of a drug delivery pump implanted under the skin of the animal or human patient, wherein the pump delivers a predetermined amount of drug for a predetermined period of time, such as 30 minutes, 90 minutes, or a time period spanning the length of the treatment regimen. The antibodies can also be administered by intravenous infusion, as a bolus or more preferably, over an extended period of time (e.g., minutes to hours).

The antibodies can be administered over a range of doses, depending on the severity of illness and titer of germline antibody or number of germline antibody producing or expressing B cells present in the patient. Doses typically range from about 2.5 to about 3000 mg/m², or more preferably, the dose of antibody administered is from about 25 to 1000 mg/m², or in particular, about 75, 150, 300 or 600 mg/m². in certain instances, antibodies can be administered in an amount of 10-375 mg/m² per week for four weeks, or 0.4-20 mg/kg per week for 2 to 10 weeks.

In additional aspects, the antibody can be administered at a dose of from about 0.01 mg/kg to about 100 mg/kg, and more preferably the dose of antibody administered is from about 0.25 mg/kg to about 20 mg/kg, or more particularly at about 1.25, 2.5, 5, 10, or 20 mg/kg. When an anti-CDIM antibody is administered, it is typically administered on a weekly basis, and in some embodiments, more frequently than once per week, as often as once per day. Administered anti-VH4-34 antibody is preferably administered over a range of dose levels from about 0.01 mg/kg up to about 20 mg/kg body weight.

It is to be understood that while the invention has been described in conjunction with the preferred specific embodiments thereof, that the description above as well as the examples that follow are intended to illustrate and not limit the scope of the invention. The practice of the present invention will employ, unless otherwise indicated, conventional techniques of organic chemistry, polymer chemistry, immunochemistry, biochemistry and the like, which are within the skill of the art. Other aspects, advantages and modifications within the scope of the invention will be apparent to those skilled in the art to which the invention pertains. Such techniques are explained fully in the literature.

All patents, patent applications, and publications mentioned herein, both supra and infra, are hereby incorporated by reference.

In the following examples, efforts have been made to ensure accuracy with respect to numbers used (e.g., amounts, temperature, etc.) but some experimental error and deviation should be accounted for. Unless indicated otherwise, temperature is in degrees ° C. and pressure is at or near atmospheric.

Abbreviations:
PMBC Peripheral blood mononuclear cells
SLE Systemic lupus erythematosis
MZ Marginal zone
Ig Immunoglobulin
ELISA Enzyme linked immunosorbent assay
RF Rheumatoid Factor
HCV Hepatitis C virus
HIV Human immunodeficiency virus
FR1 Framework 1 region

### EXAMPLE 1

### Removal of VH4-34 Abs Reduces Symptoms of Autoimmunity

Plasma or purified IgG containing a high titer of VH4-34 anti-double stranded DNA antibody from a human with SLE is injected into a mouse. The mouse is monitored carefully for symptoms of autoimmunity. An identical sample of plasma or purified IgG is treated to deplete the plasma or purified IgG of VH4-34 antibodies by passing them over a 9G4-affinity column (e.g., 9G4-sepharose). An identical amount of antibody depleted of VH4-34 is injected into a second mouse, and the mouse is monitored carefully for symptoms of autoimmune disease. The outcome for the mouse treated with the VH4-34 antibody containing sample is compared with the outcome for the mouse treated with the VH4-34 antibody depleted sample for evidence of reduction in symptoms of autoimmune disease following the depletion procedure.

### EXAMPLE 2

### Removal of VH4-34 Antibody Expressing Cells by Anti-VH4-34 Antibodies

Peripheral blood mononuclear cells (PMBC) are isolated and cultured from a patient having a high titer of VH4-34 anti-DNA antibodies, for example a patient suffering from SLE. The cells are treated with 9G4 (or other antibody having specific binding for VH4-34 antibodies) or a control antibody, in the presence of complement. The titer of anti-DNA antibodies in the supernatants of the cultured PMBC is measured to determine if the titer decreases in cells treated with anti-VH4-34 antibody (e.g., 9G4) relative to the control antibody. The titer is observed over a period of several days. The cultures are assayed for the number of 9G4+ CD19+ cells to determine if there is a correlation between depletion of 9G4+ CD19+ cells and the decrease in anti-DNA antibodies in the culture supernatant.

### EXAMPLE 3

### Treatment of a Patient with Cold Agglutinin Disease with a Therapeutic Amount of an Anti-VH4-34 Antibody

A patient suffering from cold agglutinin disease wherein the hemolytic autoantibodies are germline antibodies derived from the VH4-34 gene locus is administered a therapeutic dose of an anti-VH4-34 antibody, preferably a cytolytic humanized version of the 9G4 antibody, either intravenously or by some other parenteral route. Following administration of the antibody the patient's autoantibody producing VH4-34 B-cell population is eliminated or reduced, and the production of pathologic autoantibodies is eliminated or reduced, resulting in clinical benefit to the patient manifested as a decrease in cold-induced hemolysis, and improvement or resolution of the patient's resulting anemia. The patient may require multiple treatments with the anti-VH4-34 antibody to achieve response or durable remission. The administered anti-VH4-34 antibody may be administered over a range of dose levels, for example, from 0.01 mg/kg up to 20 mg/kg body weight.

### EXAMPLE 4

### Treatment of a Patient with Systemic Lupus Erythematosus with a Therapeutic Amount of an Anti-VH4-34 Antibody

A patient suffering from systemic lupus erythematosus wherein the patient has circulating pathologic germline autoantibodies derived from the VH4-34 gene locus is administered a therapeutic dose of an anti-VH4-34 antibody, preferably a cytolytic humanized version of the 9G4 antibody, either intravenously or by some other parenteral route. Following administration of the antibody the patient's autoantibody producing VH4-34 B-cell population is eliminated or reduced, and the production of pathologic autoantibodies is eliminated or reduced, resulting in clinical benefit to the patient manifested as a decrease in or complete resolution of the patient's signs and symptoms of systemic lupus erythematosus. The patient may require multiple treatments with the anti-VH4-34 antibody to achieve response or durable remission. The administered anti-VH4-34 antibody may be administered over a range of dose levels, for example, from 0.01 mg/kg up to 20 mg/kg body weight.

### EXAMPLE 5

### Treatment of a Patient with a VH4-34 Germline Antibody Expressing B-cell Cancer with a Therapeutic Amount of an Anti-VH4-34 Antibody

A patient suffering from a B-cell cancer such as acute lymphoblastic leukemia, chronic lymphcytic leukemia, Hodgkin's lymphoma, or non-Hodgkins lymphoma, wherein the patient's cancerous B-cells express germline antibody derived from the VH4-34 gene locus, is administered a therapeutic dose of an anti-VH4-34 antibody, preferably a cytolytic humanized version of the 9G4 antibody, either intravenously or by some other parenteral route. Following administration of the antibody the patient's cancerous B-cell population is eliminated or reduced, resulting in clinical benefit to the patient manifested as a reduction in pathologic signs and symptoms associated with the cancer, or a complete and durable remission of all signs and symptoms associated with the cancer. The patient may require multiple treatments with the anti-VH4-34 antibody to achieve response or durable remission. The administered anti-VH4-34 antibody may be administered over a range of dose levels, for example, from 0.01 mg/kg up to 20 mg/kg body weight.

### EXAMPLE 6

### Treatment of a Patient with Cold Agglutinin Disease with an Immunosorbent Specific for VH4-34 Antibodies to Remove Pathologic Antibodies

A patient suffering from cold agglutinin disease wherein the patient has circulating pathologic germline autoantibodies derived from the VH4-34 gene locus is treated by passing the patient's blood or plasma over an immunoadsorbent specific for VH4-34 antibodies, to remove pathologic antibodies and pathologic antibody producing B-cells from the patient's blood or plasma. Following the plasmapheresis or leukopheresis procedure, the patient also may be administered a therapeutic dose of an anti-VH4-34 antibody, preferably a cytolytic humanized version of the 9G4 antibody, either intravenously or by some other parenteral route. Administration of the cytolytic anti-VH4-34 antibody in sequence following the removal of circulating VH4-34 antibodies by the plasmapheresis or leukopheresis procedure reduces or eliminates the risk of inducing formation of immune complexes and associated adverse clinical events in the course of administering the therapeutic cytolytic anti-VH4-34 antibody. Following administration of the therapeutic antibody the patient's autoantibody producing VH4-34 B-cell population is eliminated or reduced, and the production of pathologic autoantibodies is eliminated or reduced, resulting in clinical benefit to the patient manifested as a decrease in cold-induced hemolysis, and improvement or resolution of the patient's resulting anemia. The patient may require multiple treatments with plasmapheresis or leukopheresis using the immunosorbent specific for VH4-34 antibodies, and multiple treatments with the therapeutic anti-VH4-34 antibody to achieve response or durable remission. The administered anti-VH4-34 antibody may be administered over a range of dose levels, for example, from 0.01 mg/kg up to 20 mg/kg body weight.

### EXAMPLE 7

### Treatment of a Patient with Systemic Lupus Erythematosus with an Immunosorbent Specific for VH4-34 Antibodies to Remove Pathologic Antibodies

A patient suffering from systemic lupus erythematosus wherein the hemolytic autoantibodies are gernline antibodies derived from the VH4-34 gene locus is treated by passing the patient's blood or plasma over an immunoadsorbent specific for VH4-34 antibodies, to remove pathologic antibodies and pathologic antibody producing B-cells from the patient's blood or plasma. Following the plasmapheresis or leukopheresis procedure, the patient also may be administered a therapeutic dose of an anti-VH4-34 antibody, preferably a cytolytic humanized version of the 9G4 antibody, either intravenously or by some other parenteral Administration of the cytolytic anti-VH4-34 antibody in sequence following the removal of circulating VH4-34 antibodies by the plasmapheresis or leukopheresis procedure reduces or eliminates the risk of inducing formation of immune complexes and associated adverse clinical events in the course of administering the therapeutic cytolytic anti-VH4-34 antibody. Following administration of the therapeutic antibody the patient's autoantibody producing VH4-34 B-cell population is eliminated or reduced, and the production of pathologic autoantibodies is eliminated or reduced, resulting in clinical benefit to the patient manifested as a decrease or complete resolution of the patient's signs and symptoms of systemic lupus erythematosus. The patient may require multiple treatments with plasmapheresis or leukopheresis using the immunosorbent specific for VH4-34 antibodies, and multiple treatments with the therapeutic anti-VH4-34 antibody to achieve response or durable remission. The administered anti-VH4-34 antibody may be administered over a range of dose levels, for example, from 0.01 mg/kg up to 20 mg/kg body weight

### EXAMPLE 8 cl Ex-vivo Purging of the Bone Marrow of a Patient Suffering from an Autoimmune Disease or B Cell Cancer of Pathologic Cells Prior to Reimplantation of the Bone Marrow

A patient suffering from an autoimmune disease manifested by production of germline VH4-34 derived autoantibodies, or a B-cell cancer expressing germline VH4-34 antibody, undergoes harvest of bone marrow for autologous transplantation. The bone marrow is treated ex-vivo with a therapeutic amount of a a cytolytic anti-VH4-34 antibody, preferably a complement fixing humanized version of the 9G4 antibody, in the presence of complement, resulting in the purging of the bone marrow of the autoimmune disease causing B-cells, or the cancerous B-cell population. Following myeloablative therapy, the patient is administered their autologous purged bone marrow, resulting in reconstitution of the patient's normal bone marrow finction, free of production of pathologic VH4-34 autoantibodies or free of the B-cell cancer population, resulting in clinical benefit to the patient.

### EXAMPLE 9

### Monitoring the Efficacy of a Therapeutic Treatment in a Patient Suffering from an Autoimmune Disease or B-cell Cancer Secreting or Expressing a VH4-34 Antibody

The efficacy of a therapeutic treatment in a patient suffering from an autoimmune disease or a B-cell cancer secreting or expressing a VH4-34 antibody, is monitored by obtaining serial samples of blood or bone marrow or lymphoid tissue from the patient, contacting said sample with an amount of anti-VH4-34 antibody sufficient to bind to VH4-34 antibodies present in the sample of blood or bone marrow or lymphoid tissue, determining the amount of anti-VH4-34 antibody bound in the sample of blood or bone marrow or lymphoid tissue, and correlating the amount of anti-VH4-34 antibody bound with the efficacy of treatment to reduce the number of VH4-34 antibody producing or cell surface expressing B cells or the amount of VH4-34 antibody in the sample of blood or bone marrow or lymphoid tissue obtained from the patient at a time period prior to initiation of the therapeutic treatment. Said determinations of the amounts of VH4-34 antibodies or VH4-34 antibody producing B-cells may be performed by ELISA, RIA, flow cytometry, immunohistochemistry, or other quantitative or qualitative analytical methods. Over the time course of an individual patient's disease, information obtained by the monitoring procedure will be used to determine relapse of the patient's autoimmune disease or B-cell cancer, and to determine the appropriate time to repeat or add new therapeutic interventions for the patient's autoimmune disease or B-cell cancer.

What is claimed is:

1. A method for reducing the amount of B cells or plasma cells producing pathologic antibodies in the body of a patient suffering from an autoimmune disease, comprising treating the patient with a therapeutically effective dose of an antibody having specific binding for an epitope present on germline antibodies.

2. The method of claim 1, wherein the germline antibodies are selected from VH4-34, VH1-69, V71-2, V71-4, VH4-18, VH72-1, or V2-1 antibodies.

3. The method of claim 1, wherein the antibody having specific binding for an epitope present on gernline antibodies is 9G4, humanized 9G4, chimerized 9G4, or fragments or conjugates thereof.

4. The method of claim 1, wherein the antibody having specific binding for an epitope present on germline antibodies is 9G4, G6, 17.109, or LC1, humanized 9G4, G6, 17.109, or LC1, chimerized 9G4, G6, 17.109, or LC1, or fragments or conjugates thereof.

5. The method of claim 1, further comprising treating the patient with an additional pharmaceutically active agent, therapeutically effective treatment or other adjunct therapy.

6. The method of claim 5, wherein the additional pharmaceutically active agent is a chemotherapeutic agent, complement activation inhibitor, antimetabolite, steroid, toleragen, anti-B cell agent, anti-T cell agent, anticoagulant or intravenous immunoglobulin.

7. A method for reducing the amount of VH4-34 antibody producing B cells or plasma cells in a patient suffering from an autoimmune disease, comprising administering a therapeutically effective amount of an antibody having specific binding for an epitope present on VH4-34 antibodies.

8. The method of claim 7, wherein the antibody having specific binding for an epitope present on VH4-34 antibodies is 9G4, humanized or chimerized 9G4, or fragments or conjugates thereof.

9. A method for treating a patient suffering from a B cell cancer expressing cell surface germline antibodies, comprising treating the patient with a therapeutically effective dose of an antibody having specific binding for an epitope present on germline antibodies.

10. The method of claim 9, wherein the germline antibodies are selected from VH4-34, VH1-69, V71-2, V71-4, VH4-18, VH72-1, or V2-1 antibodies.

11. The method of claim 9, wherein the germline antibodies are selected from VH4-34 antibodies.

12. The method of claim 9, wherein the antibody having specific binding for an epitope present on germline antibodies is 9G4, humanized or chimerized 9G4, or fragments or conjugates thereof.

13. The method of claim 9, wherein the antibody having specific binding for an epitope present on germline antibodies is 9G4, G6, 17.109, or LC1, humanized 9G4, G6, 17.109, or LC1, chimerized 9G4, G6, 17.109, or LC1, or fragments or conjugates thereof.

14. The method of claim 9, further comprising treating the patient with an additional pharmaceutically active agent selected from a chemotherapeutic agent, anti-B cell agent, cell growth regulator and/or inhibitor, immune modulator or combinations thereof.

15. The method of claim 14, wherein the chemotherapeutic agent is asparaginase, epipodophyllotoxin, camptothecin, antibiotic, platinum coordination complex, alkylating agent, folic acid analog, pyrimidine analog, purine analog, topoisomerase inhibitor, or an agent that disrupts the cytoskeleton, or mixtures thereof.

16. The method of claim 14, wherein the anti-B cell agent is selected from antibodies or inhibitors of CD11a, CD19, CD20, CD21, CD22, CD25, CD34, CD37, CD38, CD40, CD45, CD52, CD80, CD 86, IL-4R, IL-6R, IL-8R, IL-13, IL-13R, α-4/β-1 integrin (VLA4), BLYS receptor, cell surface idiotypic Ig, CDIM, tumor necrosis factor (TNF), or combinations thereof.

17. The method of claim 16, wherein the anti-B cell agent is an anti-CDIM antibody.

18. The method of claim 17, wherein the anti-CDIM antibody is selected from mAb 216, RT-2B, FS 12, A6(H4C5), Cal-4G, S20A2, FS 3, Gee, HT, Z2D2, or Y2K.

19. A method for purging the bone marrow of a patient suffering from autoimmune disease or B cell cancer prior to reimplantation of the bone marrow in the patient after myeloablative therapy, comprising treating the bone marrow of a patient ex vivo with a therapeutically effective amount of an antibody having specific binding for an epitope present on germline antibodies.

20. The method of claim 19, further comprising treating the bone marrow with an additional pharmaceutically active agent.

21. The method of claim 19, wherein the antibody having specific binding for an epitope present on germline antibodies is 9G4, humanized or chimerized 9G4, or fragments or conjugates thereof.

22. The method of claim 19, wherein the antibody having specific binding for an epitope present on germline antibodies is 9G4, G6, 17.109, or LC1, humanized 9G4, G6, 17.109, or LC1, chimerized 9G4, G6, 17.109, or LC1, or fragments or conjugates thereof.

23. A method for purging the bone marrow of a patient suffering from autoimmune disease or B cell cancer prior to reimplantation of the bone marrow in the patient after myeloablative therapy, comprising treating the bone marrow of a patient ex vivo with a therapeutically effective amount of an antibody having specific binding for an epitope present on VH4-34 antibodies.

24. The method of claim 23, further comprising treating the bone marrow with an additional pharmaceutically active agent.

25. The method of claim 23, wherein the antibody having specific binding for an epitope present on VH4-34 antibodies is 9G4, humanized or chimerized 9G4, or fragments or conjugates thereof.

26. A method for treating a patient suffering from autoimmune disease or a B cell cancer, comprising treating the patient with a therapeutically effective amount of an antibody having specific binding for an epitope present on germline antibodies, and further comprising treating the patient with a therapeutically effective amount of an anti-B cell agent.

27. The method of claim 26, wherein the anti-B cell agent is an anti-CDIM antibody.

28. The method of claim 27, wherein the anti-CDIM antibody is selected from mAb 216, RT-2B, FS 12, A6(H4C5), Cal-4G, S20A2, FS 3, Gee, HT, Z2D2, or Y2K.

29. The method of claim 27, wherein sufficient time is provided to allow the antibody having specific binding for an epitope present on germline antibodies to clear from the plasma of the patient prior to administering the anti-CDIM antibody.

30. The method of claim 27, wherein sufficient time is provided to allow the anti-CDIM antibody to clear from the plasma of the patient prior to administering the antibody having specific binding for an epitope present on germline antibodies.

31. A method for removing pathologic antibodies from the body of a patient suffering from autoimmune disease, comprising contacting the blood or plasma of the patient with an immunoadsorbent having specific binding for an epitope present on germline antibodies.

32. The method of claim 31, wherein the immunoadsorbent having specific binding for an epitope present on VH4-34 antibodies comprises 9G4, G6, 17.109, or LC1, humanized 9G4, G6, 17.109, or LC1, chimerized 9G4, G6, 17.109, or LC1, or fragments or conjugates thereof.

33. The method of claim 31, wherein said contacting results in a reduction in the amount of germline antibodies present in the patient.

34. The method of claim 31, wherein said contacting results in a reduction in the number of cells expressing germline antibodies in the patient.

35. The method of claim 31, wherein said germline antibodies are selected from VH4-34, VH1-69, V71-2, V71-4, VH4-18, VH72-1, or V2-1 antibodies.

36. A method for removing pathologic antibodies from the body of a patient suffering from autoimmune disease, comprising contacting the blood or plasma of the patient with an immunoadsorbent having specific binding for an epitope present on VH4-34 antibodies, wherein said contacting results in the reduction in the amount of VH4-34 antibodies present in the blood or plasma of the patient.

37. The method of claim 36, wherein the immunoadsorbent having specific binding for an epitope present on VH4-34 antibodies comprises 9G4, humanized or chimerized 9G4, or fragments or conjugates thereof.

38. A method for reducing the number of VH4-34 antibody producing B cells or plasma cells in a patient suffering from an autoimmune disease, comprising contacting the blood or plasma of the patient with an immunoadsorbent having specific binding for an epitope present on VH4-34 antibodies, wherein said contacting results in the reduction in the amount of VH4-34 antibody producing B cells present in the blood, lymphoid tissues or bone marrow of the patient.

39. The method of claim 38, wherein the immunoadsorbent having specific binding for an epitope present on VH4-34 antibodies comprises 9G4, humanized or chimerized 9G4, or fragments or conjugates thereof.

40. A method for treating a patient suffering from a B cell cancer expressing cell surface germline antibody, comprising contacting the blood of the patient with an immunoadsorbent having specific binding for an epitope present on germline antibodies, wherein said contacting results in the reduction in the amount of germline antibody expressing B cell cancer cells present in the blood, lymphoid tissues or bone marrow of the patient.

41. The method of claim 40, further comprising administering a therapeutically effective amount of an antibody having specific binding for an epitope present on germline antibodies to the patient.

42. The method of claim 40, wherein the antibody having specific binding for an epitope present on germline antibodies is 9G4, humanized or chimerized 9G4, or fragments or conjugates thereof.

43. The method of claim 40, wherein the antibody having specific binding for an epitope present on germline antibodies is 9G4, G6, 17.109, or LC1, humanized 9G4, G6, 17.109, or LC1, chimerized 9G4, G6, 17.109, or LC1, or fragments or conjugates thereof.

44. The method of claim 40, further comprising administering a therapeutically effective amount of an anti-CDIM antibody to the patient.

45. The method of claim 44, wherein the anti-CDIM antibody is selected from mAb 216, RT-2B, FS 12, A6(H4C5), Cal-4G, S20A2, FS 3, Gee, HT, Z2D2, or Y2K.

46. A method for treating a patient suffering from a B cell cancer expressing cell surface VH4-34 antibody, comprising contacting the blood of the patient with an immunoadsorbent having specific binding for an epitope present on VH4-34 antibodies, wherein said contacting results in the reduction in the amount of VH4-34 antibody expressing B cell cancer cells present in the blood, lymphoid tissues or bone marrow of the patient.

47. The method of claim 46, further comprising administering a therapeutically effective amount of an antibody having specific binding for an epitope present on VH4-34 antibodies to the patient.

48. The method of claim 47, wherein the antibody having specific binding for an epitope present on VH4-34 antibodies is 9G4, humanized or chimerized 9G4, or fragments or conjugates thereof.

49. The method of claim 47, further comprising administering a therapeutically effective amount of an anti-CDIM antibody to the patient.

50. The method of claim 49, wherein the anti-CDIM antibody is selected from mAb 216, RT-2B, FS 12, A6(H4C5), Cal-4G, S20A2, FS 3, Gee, HT, Z2D2, or Y2K.

51. A method for monitoring the efficacy of a therapeutic treatment in a patient suffering from an autoimmune disease or a B cell cancer, comprising obtaining a sample of blood or bone marrow or lymphoid tissue from the patient, contacting said sample with an amount of anti-germline antibody sufficient to bind to germline antibodies present in the sample of blood or bone marrow or lymphoid tissue, determining the amount of anti-germline antibody bound in the sample of blood or bone marrow or lymphoid tissue, and correlating the amount of anti-germline antibody bound with the efficacy of treatment to reduce the number of germline antibody producing or cell surface expressing B cells or the amount of germline antibody in the sample of blood or bone marrow or lymphoid tissue obtained from the patient at a time prior to initiation of the therapeutic treatment.

52. The method of claim 51, wherein the anti-germline antibody is 9G4, humanized or chimerized 9G4, or fragments or conjugates thereof.

53. The method of claim 51, wherein the anti-germline antibody is 9G4, G6, 17.109, or LC1, humanized 9G4, G6, 17.109, or LC1, chimerized 9G4, G6, 17.109, or LC1, or fragments or conjugates thereof.

54. The method of claim 51, wherein the anti-germline antibody is associated with a substrate for performing an assay selected from ELISA or radioimmunoassay.

55. The method of claim 51, wherein the anti-germline antibody is utilized in flow cytometry.

56. The method of claim 51, wherein the therapeutic treatment is plasmapheresis, leukopheresis, or treatment with an anti-germline antibody or additional pharmaceutically active agent comprising an anti-B cell agent, anti-T cell agent, chemotherapeutic agent, toleragen, complement activation inhibitor, antimetabolite, steroid, anticoagulant or intravenous immunoglobulin, or combinations thereof.

57. A method for monitoring the efficacy of a therapeutic treatment in a patient suffering from an autoimmune disease or a B cell cancer, comprising obtaining a sample of blood or bone marrow or lymphoid tissue from the patient, contacting said sample with an amount of anti-VH4-34 antibody sufficient to bind to VH4-34 antibodies present in the sample of blood or bone marrow or lymphoid tissue, determining the amount of anti-VH4-34 antibody bound in the sample of blood or bone marrow or lymphoid tissue, and correlating the amount of anti-VH4-34 antibody bound with the efficacy of treatment to reduce the number of VH4-34 antibody producing or cell surface expressing B cells or the amount of VH4-34 antibody in the sample of blood or bone marrow or lymphoid tissue obtained from the patient at a time period prior to initiation of the therapeutic treatment.

58. The method of claim 57, wherein the anti-VH4-34 antibody is 9G4, humanized or chimerized 9G4, or fragments or conjugates thereof.

59. The method of claim 57, wherein the anti-VH4-34 antibody is associated with a substrate for performing an assay selected from ELISA or radioimmunoassay.

60. The method of claim 57, wherein the anti-VH4-34 antibody is utilized in flow cytometry.

61. The method of claim 57, wherein the therapeutic treatment is plasmapheresis, leukopheresis, or treatment with an additional pharmaceutically active agent comprising an anti-B cell agent, anti-T cell agent, chemotherapeutic agent, toleragen, complement activation inhibitor, antimetabolite, steroid, anticoagulant or intravenous immunoglobulin

62. A kit for use in monitoring therapeutic response in a patient in need thereof to administration of a treatment for autoimmune disease or B cell cancer, comprising an amount of anti-germline antibody effective to bind to germline antibodies present in a sample of blood or bone marrow or lymphoid tissue from a patient suffering from said autoimmune disease or B cell cancer.

63. A kit for use in monitoring the therapeutic response in a patient in need thereof to administration of a treatment for autoimmune disease or B cell cancer, comprising an amount of anti-VH4-34 antibody effective to bind to VH4-34 antibodies present in a sample of blood or bone marrow or lymphoid tissue from a patient suffering from said autoimmune disease or B cell cancer.

64. A kit for use in monitoring the therapeutic response in a patient in need thereof to administration of a treatment for autoimmune disease or B cell cancer, comprising an amount of 9G4, humanized or chimerized 9G4, or fragments or conjugates thereof, effective to bind to VH4-34 antibodies present in a sample of blood or bone marrow or lymphoid tissue from a patient suffering from said autoimmune disease or B cell cancer.

65. An immunoadsorbent for use in plasmapheresis and leukopheresis, comprising an anti-germline antibody or fragment thereof associated with a sorbent suitable for use in a plasmapheresis or leukopheresis apparatus.

66. The immunoadsorbent of claim 65, wherein the anti-germline antibody is selected from an antibody having specific binding for VH4-34, VH1-69, V71-2, V71-4, VH4-18, VH72-1, or V2-1 antibodies.

67. The immunoadsorbent of claim 65, wherein the anti-germline antibody is an anti-VH4-34 antibody.

68. The immunoadsorbent of claim 67, wherein the anti-VH4-34 antibody is 9G4, humanized or chimerized 9G4, or fragments or conjugates thereof.

69. The immunoadsorbent of claim 67, wherein the anti-VH4-34 antibody is 9G4, G6, 17.109, or LC1, humanized 9G4, G6, 17.109, or LC1, chimerized 9G4, G6, 17.109, or LC1, or fragments or conjugates thereof.

70. A method for treating a patient suffering from cold agglutinin disease, comprising treating the patient with a therapeutically effective dose of an antibody having specific binding for an epitope present on germline antibodies.

71. The method of claim 70, wherein the germline antibodies are selected from VH4-34, VH1-69, V71-2, V71-4, VH4-18, VH72-1, or V2-1 antibodies.

72. The method of claim 71, wherein the germline antibodies are VH4-34 antibodies.

73. The method of claim 70, wherein the antibody having specific binding for an epitope present on germline antibodies is 9G4, humanized or chimerized 9G4, or fragments or conjugates thereof.

74. A pharmaceutical composition comprising a therapeutically effective dose of an antibody having specific binding for an epitope present on germline antibodies for the treatment of autoimmune disease or B cell cancer in a human patient.

75. The pharmaceutical composition of claim 74, wherein the therapeutically effective dose of antibody is effective to reduce the amount of B cells or plasma cells producing pathologic antibodies in the body of the patient.

76. The pharmaceutical composition of claim 74, wherein the therapeutically effective dose of antibody is effective to reduce the amount of B cells or plasma cells expressing or producing germline antibodies in the body of the patient.

77. The pharmaceutical composition of claim 74, wherein the therapeutically effective dose of antibody is effective to reduce the amount of pathologic antibodies in the body of the patient.

78. The pharmaceutical composition of claim 74, wherein said germline antibodies are selected from VH4-34, VH1-69, V71-2, V71-4, VH4-18, VH72-1, or V2-1 antibodies.

79. The pharmaceutical composition of claim 78, wherein the antibody having specific binding for an epitope present on VH4-34 antibodies comprises 9G4, G6, 17.109, or LC1, humanized 9G4, G6, 17.109, or LC1, chimerized 9G4, G6, 17.109, or LC1, or fragments or conjugates thereof.

80. The pharmaceutical composition of claim 74, wherein the therapeutically effective dose of antibody is effective to reduce the amount of germline antibodies present in the body of the patient.

81. The pharmaceutical composition of claim 74, wherein the therapeutically effective dose of antibody is effective to reduce the amount of cold agglutinins present in the body of the patient.
